(19) United States
(12) Patent Application Publication
(10) Pub. No.: US 2023/0365625 A1
(43) Pub. Date: Nov. 16, 2023

(54) METHODS FOR TREATING AND/OR PREVENTING UNDESIRABLE SEQULAE OF NASAL TURBINOPLASTY OR FUNCTIONAL ENDOSCOPIC SINUS SURGERY

(71) Applicant: 3-D Matrix, Ltd., Cambridge, MA (US)

(72) Inventors: Lisa Spirio, S. Weymouth, MA (US); Michael Fook-Ho Lee, Marsfield (AU); Jennifer Fong Ha, Bull Creek (AU); Maurice Bagot D'Arc, Paris (FR)

(73) Assignee: 3-D Matrix, Ltd., Cambridge, MA (US)

(21) Appl. No.: 18/317,075

(22) Filed: May 13, 2023

Related U.S. Application Data
(60) Provisional application No. 63/341,447, filed on May 13, 2022.

(51) Int. Cl. C07K 7/08; A61M 25/09

(52) U.S. Cl. CPC C07K 7/08; A61M 25/09; A61M 2025/091; A61M 2210/0618

(57) ABSTRACT

Endoscopic surgery targeted at the sinuses and nasal turbinates assists in managing the symptoms of chronic rhinosinusitis and other conditions. With contemporary techniques, post-operative hemorrhage, synechiae formation and poor wound healing are amongst the most common complications. The present disclosure provides the use of self-assembling peptide matrix, PuraSinus, and significantly reduces the incidence of these post-operative complications, including prevention of synechiae formation and aiding hemostasis.

### PRIORITY

This application claims priority to U.S. provisional Application No. 63/341,447, filed May 13, 2022, the entire contents of which are hereby incorporated by reference.

### SEQUENCE LISTING

The instant application contains an XML Sequence Listing which has been submitted electronically and is hereby incorporated by reference in its entirety. The Sequence Listing, created on Apr. 12, 2023, is named 3DM-21-01-TRB-US_SL.xml and is 30,517 bytes in size.

### FIELD OF THE INVENTION

The invention relates to methods for treating traumatized tissue in the nasal cavity to promote rapid healing and inhibit formation of intranasal adhesions (so-called “synechiae”). More specifically, the invention relates to a medical gel comprised of self-assembling peptides applied intraoperatively to nasal turbinates to inhibit synechiae formation and promote more rapid hemostasis, as well as to promote more rapid and complete wound healing.

### BACKGROUND OF THE INVENTION

Endoscopic surgery targeted at the sinuses and nasal turbinates assists in managing the symptoms of chronic rhinosinusitis. Turbinoplasty may also be performed in the context of cosmetic rhinoplasty. With contemporary techniques, post-operative hemorrhage, synechiae formation and poor wound healing are amongst the most common complications. The present disclosure is based, at least in part, on the finding that in a sheep model of chronic rhinosinusitis, the application of a self-assembling peptide RADA16 matrix dramatically reduces the incidence of these post-operative complications. A previous report using RADA16 in humans has suggested its efficacy in nasal surgery for this purpose with RADA (M. F. Lee et al., “A novel hemostatic agent based on self-assembling peptides [RADA16] in the setting of nasal endoscopic surgery, a case series,” Int. J. Surg. Case Rep. 41: 461-464 (2017)). An additional study involving a single human patient successfully applied RADA16 intraoperatively to prevent readhesion of the patient's surgically separated nasopharyngeal/palatal stenosis resulting from radiotherapy (E. Wong et al., Int. J. Surg. Case Rep. 70: 227-229 (2020)). However, these human studies, while experimental did not conclusively establish the efficacy and the mechanism of action of RADA16, in particularly, due to the absence of post-mortem tissue histochemistry unavailable in human studies. Additionally, while these studies addressed turbinoplasty, they did they not address use of RADA16 (SEQ ID NO:1) in other nasal surgery procedures such sinus surgeries and septoplasties, nor the use of other SAPs for any type of endoscopic intranasal surgery.

Over the past few decades, the innovation of endoscopic nasal surgery targeting the turbinates and sinuses has become a cornerstone in managing chronic rhinosinusitis refractory to medical therapy (R. Giger et al., Am. J. Rhinol. 17(6):327-333 (2003)).

Currently, the use of tamponade through the insertion of nasal packing is the “gold standard” in the US and many other jurisdictions, but this comes at the cost of the patient's comfort. Hemostatic agents using gelatin matrix-thrombin tissue and chitosan gel have been validated for use in endoscopic sinus surgery (R. K. Chandra et al., Am. J. Rhinol. 17(1): 51-5 (2003); R. Valentine et al., Am. J. Rhinol. Allergy 24(1): 70-75 (2010)). However, nasal packing and current hemostatic agents have been associated with increased rates of adhesion formation (M. S. Maccabee et al., Am. J. Rhinol. (2003) 17(4): 203-207; R. K. Chandra, et al., Am. J. Rhinol. (2005), 19(3): 240-3).

Therefore, there remains a need to develop new and improved methods for managing of negative sequelae of nasal surgeries, and in particular, reducing or preventing synechiae in turbinoplasty.

### SUMMARY OF THE INVENTION

The invention is based in part on the following study. Thirty healthy sheep underwent surgery between 2018 and 2019 to create uniform nasal mucosal injuries on the middle turbinate and opposing nasal septum. Commercially available versions of a self-assembling peptide (RADA16; PuraSinus; 3dmatrix.com/us/products/purasinus/) and a gelatin/human thrombin product (FLOSEAL) and untreated control sites were randomized to alternate injuries. Primary outcomes of intra-operative hemostasis were recorded, with synechiae formation and wound healing evaluated at 2 weeks under sedation and 6 weeks via post-mortem examination in situ and histology.

Intra-operative hemostasis time improved with RADA16 and Gelatin-thrombin (FLOSEAL) versus Control wounds (139.7±56.2 s, 145.4±58.1 s, and 224.0±69.9 s, respectively; p<0.0001 for both comparisons). Two-week synechiae scores (maximum 4 points) were similar in Controls (2.9±1.8 points) and Gelatin-thrombin (3.1±1.6) wounds (p>0.05), but were reduced in RADA16 sites by 91% versus Controls and 92% versus Gelatin-thrombin treatment (0.3±0.6 points; p<0.0001 for both comparisons). Six-week synechiae scores were similar in Control (1.1±1.7) and Gelatin-thrombin (1.7±2.0 points) wounds (p>0.05), but reduced 100% in RADA16-treated wounds. Synechiae occurred in fewer RADA16-treated sites at 2 weeks (20%) versus Gelatin-thrombin (80%) and Controls (75%; p<0.01) and at 6 weeks (0%, 50% and 35%, respectively; p<0.01). RADA16 was associated with significantly lower 6-week histopathology scores, driven by reduced submucosal fibrosis and angiogenesis.

For this sheep model of nasal surgery, the use of both PuraSinus and FLOSEAL is associated with improved hemostasis as compared to the control. In the PuraSinus group alone, there was significantly reduced synechiae formation at both 2 and 6 weeks. Histopathological findings also suggest enhancement to mucosal regeneration in the PuraSinus group. A prospective clinical trial will be required to further verify these findings in humans (see e.g., M. F. Lee, et al., A novel hemostatic agent based on self-assembling peptides [RADA16] in the setting of nasal endoscopic surgery, a case series, Int. J. Surg. Case Rep. 41: 461-464 (2017)). The invention is further based, on the insight, that the sheep study provides an experimental basis for treatment, prevention and/or re-formation of negative sequelae of turbinoplasty using alternative self-assembling peptides that possess quite different chemical compositions, tensile properties, and gelation characteristics, particularly, under in vivo conditions. Although RADA16 and Gelatin-thrombin similarly 25 accelerated hemostasis in this sheep endoscopic sinus surgery model, only RADA16 reduced postoperative synechiae formation at 2 weeks with an absence of synechiae at 6 weeks. Histology suggested RADA16 enhanced mucosal regeneration.

This invention is further based, at least in part, on a retrospective clinical study of 94 adult human patients undergoing a variety of Functional Endoscopic Sinus Surgeries (FESS), including some patients who underwent turbinoplasty and/or septoplasty indicated for chronic rhinosinusitis (CRS), intra-operative application of RADA16-I SAP (PuraSinus; (SEQ ID NO:1) 2.5% in water) to the surgical wound resulted in effective hemostasis in the absence of other hemostatic agents and devices in most patients. The absence of adhesions, also known as synechiae, was reported in almost 90% of these cases at the second of two follow-up examinations for each of the patients. This study involved human patients with a more varied range of characteristics and sinonasal pathologies than other studies and demonstrates the utility of SAPs in inducing hemostasis, promoting wound healing, prevention of adhesions during recovery longer term (the incidence of adhesions generally increases over time post-surgery), and absence of pain and patient discomfort during recovery. Also, no revision surgeries were indicated in this study.

Accordingly, the invention provides alternative peptides such as, IEIK13, KLD12, and others, for the management of the aforementioned conditions.

In summary, in certain embodiments, the invention includes methods of reducing negative sequelae from endoscopic turbinoplasty. The method comprise applying an effective amount of one or more of the self-assembling peptide solution chosen from: a) of SEQ NO:1 at a purity of at least 65% in water or physiological buffer and/or at a concentration of between 1.5 and 3.0% and b) SEQ ID NO:3 at a major peptide purity of at least 70% in water or physiological buffer and at a concentration of total peptide concentration between 1.5%-3.5% weight/volume, said application taking place during or immediately after an endoscopic turbinoplasty wherein the SAP solution is applied via a catheter to the surgically affected area in the turbinate thereby forming a transparent, resorbable viscous gel in contact with the wound. In some embodiments, the healing over a period of weeks after the endoscopic turbinoplasty, improves in one of more the following observable metrics:
A. Reduction in epithelial erosion
B. Reduction in inflammatory infiltrate
C. Reduction in submucosal fibrosis
D. Improved angiogenesis
E. Presence/absence of seromucous glands
F. Presence/absence of goblet cells

### BRIEF DESCRIPTION OF THE FIGURES

FIG. 1 shows endoscopic images of the surgical sites treated with PuraSinus at 2 weeks.

FIG. 2 shows endoscopic images of the untreated (control) surgical sites at 2 weeks.

FIG. 3A shows endoscopic images (postmortem) of the surgical sites in situ at 6 weeks. The surgical wounds were PuraSinus-treated.

FIG. 3B shows example endoscopic images by grade.

FIG. 4A shows the histopathology of the surgical site at 6 weeks for sheep treated with PuraSinus. The scale bar is 3 mm.

FIG. 4B shows the histopathology of the surgical site at 6 weeks for sheep treated with PuraSinus. The scale bar is 500 μm.

FIG. 5A shows the histopathology of the surgical site at 6 weeks for sheep treated with FLOSEAL. The scale bar is 3 mm.

FIG. 5B show the histopathology of the surgical site at 6 weeks for sheep treated with FLOSEAL The scale bar is 500 μm.

FIG. 6A shows a flowchart of patients based on extent of surgery (Complete FESS) and outcomes.

FIG. 6B shows a flowchart of patients based on extent of surgery (Limited FESS) and outcomes.

FIG. 7 shows a comparison of time to achieving hemostasis, comparing untreated controls, RADA16- and FLOSEAL-treated samples.

### DETAILED DESCRIPTION OF THE INVENTION

Characteristics of Self-Assembling Peptides for Use in the Methods of the Invention

PuraSinus (SEQ ID NO:1) is comprised of the peptide Ac-RADARADARADARADA-CONH₂, RADA16 (SEQ ID NO:1) and potentially truncated fragments thereof, about 2.5% in water. Another SAP, IEIK13 (SEQ ID NO:2), showing different gelation characteristics as expected among SAPs having different chemical makeup has also shown improved utility in achieving hemostasis in a different type of animal model relevant to the present application. As reported by S. Katsuyama et al., Minimally Invasive Therapy & Allied Technologies 29(5): 283-292 (2020), IEIK13 (called TDM-623 in that reference) forms a stiffer gel (i.e., has a higher storage modulus, G′) compared to RADA16 (TDM-621 in that reference) when exposed to physiological conditions which correlated with improved hemostasis when the product was applied to liver punch hole injuries in pigs. While reporting statistically significant improvement in hemostasis compared to RADA16, these authors also reported the absence if inflammatory cell infiltration due to the presence of the RADA16 or IEIK13 gels in the wound following application of the SAP solutions to the respective wounds. This bodes well for other in vivo and clinical applications of RADA16 and IEIK13 including the use of IEIK13 in the prevention of undesirable sequelae in turbinoplasty and other types of endoscopic sinus surgeries.

The use of D-amino acid containing SAPs, including RADA16 and IEIK13 SAPs KLD12 (SEQ ID NO:2) and QLEL12 (SEQ ID NO:4), for intraoperative treatment of human and animal patients having undergone turbinoplasty or other types of endoscopic sinus surgery are included in the present invention, particularly where a slower rate of in vivo degradation and/or resorption may be desirable.

In a report directly comparing RADA16 (SEQ ID NO:1; TDM-621 in that paper) and IEIK13 (SEQ ID NO:3; TDM-623 in that paper), although the concentration of the IEIK13 peptide solution was not given, IEIK13 was again found to form a stiffer hydrogel matrix in vitro and in vivo when exposed to physiologic conditions (pH˜7.4 and at least a low concentration of ions such as Na⁺ and/or K⁺ (H. Masuhara et al., Anns. Thorac. Cardiovasc. Surg. 2012, 18(5): 444-451)), and IEIK13 (SEQ ID NO:3) demonstrated a statistically significant improvement compared to RADA16 (SEQ ID NO:1) in achieving hemostasis in porcine punch-hole wounds to the liver which oozed blood. Spurting wounds were excluded from that analysis as neither gel would stay in the wound (S. Katsuyama et al., “Novel, infection-free, advanced hemostatic material: physical properties and preclinical efficacy,” Minimally Invasive Therapy & Allied Technologies 29(5):283-292 (2019)). These researchers also noted that IEIK13 (SEQ ID NO:3) is easier to handle than RADA16 as it can be stored at room temperature and, like RADA16 (SEQ ID NO:1), it can be applied on the resected area through a syringe or endoscopic catheter making it suitable for use in procedures such as turbinoplasty and other endoscopic nasal surgeries. The latter feature is common to the SAPs disclosed and claimed in this patent application.

In some embodiments, the SAPs comprise a sequence of amino acid residues conforming to one or more of Formulas I-IV:

((Xaa^{neu}-Xaa⁺)ₓ(Xaa^{neu}-Xaa⁻)_{y})ₙ (I)

((Xaa^{neu}-Xaa⁻)ₓ(Xaa^{neu}-Xaa⁺)_{y})ₙ (II)

((Xaa⁺-Xaa^{neu})ₓ(Xaa⁻-Xaa^{neu})_{y})ₙ (III)

((Xaa⁻-Xaa^{neu})ₓ(Xaa⁺-Xaa^{neu})_{y})ₙ (IV)

Xaa^{neu} represents an amino acid residue having a neutral charge; Xaa⁺ represents an amino acid residue having a positive charge; Xaa⁻ represents an amino acid residue having a negative charge; x and y are integers having a value of 1, 2, 3, or 4, independently; and n is an integer having a value of 1-5.

In some embodiments, the specific peptides for use in the method of the present invention can be chosen from one or more peptides listed in the Table 1 below.

TABLE 1

| Self-assembling Peptide | SEQ ID NO: |
|---|---|
| RADARADARADARADA | SEQ ID NO: 1 |
| KLDKLDKLDKLD | SEQ ID NO: 2 |
| IEIKIEIKIEIKI | SEQ ID NO: 3 |
| QLELQLELQLEL | SEQ ID NO: 4 |
| ADARADARADARADAR | SEQ ID NO: 5 |
| RAEARAEARAEARAEA | SEQ ID NO: 6 |
| RVDVRVDVRVDVRVDV | SEQ ID NO: 7 |
| RLDLRLDLRLDLRLDL | SEQ ID NO: 8 |
| RIDIRIDIRIDIRIDI | SEQ ID NO: 9 |
| RFDFRFDFRFDFRFDF | SEQ ID NO: 10 |
| AEARAEARAEARAEAR | SEQ ID NO: 11 |
| KADAKADAKADAKADA | SEQ ID NO: 12 |
| KIDIKIDIKIDIKIDI | SEQ ID NO: 13 |
| KIEIKIEIKIEIKIEI | SEQ ID NO: 14 |
| IDIKIDIKIDIKI | SEQ ID NO: 15 |
| IEIRIEIRIEIRI | SEQ ID NO: 16 |
| LELKLELKLELKL | SEQ ID NO: 17 |
| FEFKFEFKFEFKF | SEQ ID NO: 18 |
| KLDLKLDLKLDL | SEQ ID NO: 19 |
| KLELKLELKLEL | SEQ ID NO: 20 |
| FEFRFEFRFEFRF | SEQ ID NO: 21 |
| YEYKYEYKYEYKY | SEQ ID NO: 22 |
| WEWKWEWKWEWKW | SEQ ID NO: 23 |

Peptide Concentration—In accordance with one or more embodiments, rheological properties of peptide composition as described previously (U.S. Pat. No. 10,654,893) may be controlled by selection of peptide concentration, for example as may be specifically preferred for a particular indication or use of the compositions, through selection and/or adjacent of peptide concentration.

For numerous SAPs, composition stiffness has been shown in vitro to increase substantially linearly with peptide concentration. Furthermore, as described previously (U.S. Pat. No. 10,654,893), certain peptide compositions demonstrated a shear thinning property over a critical stress level. Given that the synthetic SAPs are commercially supplied sterile in water in a pre-filled syringe, shear thinning occurs when the SAP solution is delivered from the syringe to the application site intra-operatively where the SAP comes into contact with body fluids (such as blood) whereby the SAPs delivered self-assemble into a nanofiber matrix resembling an extracellular matrix which is much stiffer than the gel inside the syringe as supplied.

In vitro, the rheological properties achieved at a particular peptide concentration vary depending on the identity of the peptide. For example, the storage modulus G′ of KLD12 (SEQ ID NO:2) 1.5% in water was found to be about 350 Pa similar to that of 2.5% RADA16 (SEQ ID NO:1) in water under the same test conditions. The storage modulus G′ of 1% IEIK13 (SEQ ID NO:3) in water (˜700 Pa) was found to be similar to that of 2.5% KLD12 (SEQ ID NO:2) in water and higher than that of 2.5% RADA16 (SEQ ID NO:1) in water (˜350 Pa) under the same test conditions (U.S. Pat. No. 10,654,893—Tables 3 and 3A). Overall, the order of rheological strength among these compositions was IEIK13 (SEQ ID NO:3)>KLD12 (SEQ ID NO:2)>RADA16 (SEQ ID NO:1), so a composition of IEIK13 (SEQ ID NO:3) showed greater rheological strength than did a composition of KLD12 (SEQ ID NO:2), which in turn showed greater rheological strength than did a composition of RADA16 (SEQ ID NO:1) when peptide concentration in water was the same in each case.

In some embodiments, peptide concentration in a peptide composition for use in accordance with the present invention is at least 0.05%, at least 0.25%, at least 0.5%, at least 0.75%, at least 1.0% or more. In some embodiments, peptide concentration in a peptide composition for use in accordance with the present invention is less than 5%, less than 4.5%, less than 4%, less than 3.5%, less than 3%, or less. In some embodiments, peptide concentration in a peptide composition for use in accordance with the present invention is within a range between about 0.5% and about 3%. In some embodiments, peptide concentration in a peptide composition for use in accordance with the present invention is within a range between about 0.5% and about 2.5%. In some embodiments, peptide concentration in a peptide composition for use in accordance with the present invention is within a range between about 1% and about 3%. In some embodiments, peptide concentration in a peptide composition for use in accordance with the present invention is within a range between about 1% and about 2.5%. In some embodiments, peptide concentration in a peptide composition for use in accordance with the present invention is about 0.5%, about 1%, about 1.5%, about 2%, about 2.5%, about 3%, or more. In some particular embodiments, where the peptide is RADA16 (SEQ ID NO:1), peptide concentration in peptide compositions of the present invention is within a range of about 0.05% to about 5%.

In some particular embodiments, where the peptide is KLD12 (SEQ ID NO:2), peptide concentration in peptide compositions of the present invention is within a range of about 0.05% to about 5%.

In some particular embodiments, where the peptide is IEIK13 (SEQ ID NO:3), peptide concentration in peptide compositions of the present invention is within a range of about 0.05% to about 2.5% in water, given that beyond this concentration in water only the peptide may be too viscous to be useful (U.S. Pat. No. 10,654,893, see Table 1 therein). However, when other components are added to the IEIK13 (SEQ ID NO:3) solution and/or attached to the peptides, e.g., biologically active peptides and/or pharmaceuticals, the rheological properties of the solution will change such that administration of higher concentrations of SAPs, e.g., 5% or more, in solution may be both useful and advantageous for various indications.

Hydrogel Formation of SAPs when Exposed to In Vivo or In Vivo-Like Conditions—In the present invention, the SAP solution is applied in the clinical setting to the surgical site via a syringe with a small aperture nozzle and/or or endoscopic catheter. When the SAP solution is applied (not injected) to the targeted site in vivo, the SAPs self-assemble at the site into nanofibers that form a hydrogel matrix, thereby causing or contributing to rapid hemostasis by occluding the vessels of the bleeding or oozing tissue, and by remaining at the site in contact with or integral with the wounded tissue for a period of time which will vary according to the type, severity, location and extent of the wound and correspondingly with the identity of the SAP or SAPs, their concentration as applied, the amount applied, and the identity, form and concentration of any other components comprising the SAPs and comprising the solution in which they are applied. This hemostatic effect does not depend on the patient's own coagulation mechanisms as shown by numerous studies, for example, in a porcine model of a liver punch biopsy using a self-assembling peptide, termed AC5 in that paper, applied to the wound was effective for hemostasis in both heparinized and non-heparinized animals (D. Csukas et al., Nanomedicine: Nanotechnology, Biology and Medicine 2015 11(8): 2025-2031).

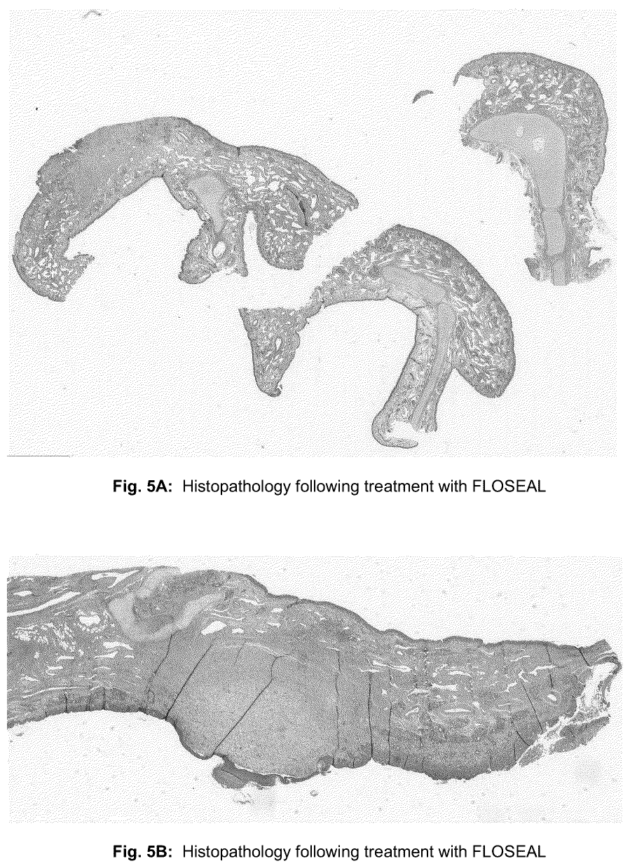

RADA16 (SEQ ID NO:1), KLD12 (SEQ ID NO:2) and IEIK13 (SEQ ID NO:3) SAPs 1% in water each showed fold increases in storage modulus G′ after treatment with DMEM (Dulbecco's Modified Eagle's Medium) to mimic in vivo conditions. The increase in G′ under the same test conditions was 43.5-, 23.9- and 7.4-fold after DMEM treatment relative to before treatment for RADA16 (SEQ ID NO:1), KLD12 (SEQ ID NO:2) and IEIK13 (SEQ ID NO:3), respectively. Of the samples treated with DMEM, IEIK13 (SEQ ID NO:3) was shown to have the highest storage modulus at 5314 Pa. (U.S. Pat. No. 10,654,893; FIGS. 5A and 5B). This increased stiffness of IEIK13 (SEQ ID NO:3) relative to RADA16 (SEQ ID NO:1) has been shown in situ (Caravasili et al., ACS Biomater. Sci. Eng. 3(12): 3386-3394 (2017)) and in vivo (S. Katsuyama et al., Minimally Invasive Therapy & Allied Technologies 29(5): 283-292 (2020)).

The medical device PuraSinus (SEQ ID NO:1) applied to the surgical site in the claimed methods is a self-assembling resorbable peptide aqueous solution currently used in post-endoscopic resection bleeding, where it has been found to be easy and safe to use while offering effective hemostasis. As a soluble and transparent gel that can be applied through a nozzle or an endoscopic catheter, the gel's ease of application lends itself to its use deep in the nasal cavity. As studies described herein show, application of PuraSinus ((SEQ ID NO:1) 3-D Matrix, Ltd., or 3-Matrix, Inc.) at the surgical site post-surgery demonstrates improved healing compared to a control group not treated with a healing agent post-turbinoplasty and compared to subjects treated with FLOSEAL (Baxter Healthcare).

Currently in Australia, PuraStat (PuraSinus or PuraGel; RADA16-I (SEQ ID NO:1)) has been approved for use in exudative hemorrhage from blood vessels and parenchyma of solid organs, vascular anastomoses and small vessels and capillary vessels of the GI tract (ARTG entry 267486). The self-assembling peptides (SAPs) disclosed herein demonstrate improved results following this type of surgery in studies developed in compliance with International Standard ISO 10993-1, “Biological evaluation of medical devices” (2016). All staff and facilities conducted the study in compliance with WHO Good Laboratory Practice.

In another embodiment of the invention, the SAP Ac-IEIKIEIKIEIKI-NH₂, IEIK13 (SEQ ID NO:3; also known as TDM-623) has demonstrated different gelation properties in vitro and achieved improved hemostasis relative to RADA16 (TDM-621 in that study; (SEQ ID NO:1)) in vivo in a pig model for punch hole injuries to the liver (Katsuyama S. et al., “Novel, infection-free, advanced hemostatic material: physical properties and preclinical efficacy,” Minimally Invasive Therapy & Allied Technologies, 29:5, 283-292 (2020)). For example, in solution (assumed to be 2.5% wt/vol SAP), the median storage modulus G′ of RADA16 reported was 384 Pa and the median loss modulus G″ reported was 24 Pa and the storage and loss moduli of IEIK13 (concentration unknown) were 1145 Pa and 116 Pa, respectively, in those studies, where the differences are statistically significant. After hydrogel formation in DMEM in vitro, the storage and loss moduli of RADA16-I (SEQ ID NO:1) and IEIK13 showed substantial increases. This observed increase is consistent with numerous other studies. In addition, the values were significantly higher after than before DMEM treatment (p<0.0001). The higher viscosity of IEIK13 compared to RADA16 ((SEQ ID NO:1); both before and after gelation) appeared to correlate with improved short-term hemostasis likely due partially to improved retention of the gel in the wound in that in vivo study. This animal study (S. Katsuyama, et al.) appears to have lasted only 5 minutes and was not intended to investigate healing or longer-term hemostasis at the wound site while still suggesting advantages of IEIK13 SAPs compared to RADA16-I (SEQ ID NO:1) SAPs in wound treatment.

In US Patent Application Publication No. 2016/0317607, additional observations of the properties of PuraSinus (RADA16—referred to in that application as PuraStat (SEQ ID NO:1)) with major peptide content over 65-70% make clear some advantages of these peptides. The tested solutions with less than 65-70% pure peptides were not observed to self-assemble into gels when exposed to about neutral pH. Accordingly, in the preferred embodiments of the present invention, at least 65%, 70%, 75%, 80%, 85% or more of the SAP being used and found in the administered solution is full-length peptide, with rest being less than full-length versions thereof, due to synthesis impurities and/or degradants of the full-length peptide.

The transparency of the above SAP hydrogels is advantageous for visualization of the tissue covered by the gel in vivo and for observation of wound healing while the nanofibrous matrix is still present. This benefit of transparency is noted in European Patent Application Pub. No. 2581097, although the disclosed wound dressing in that application is not suitable for endoscopic administration.

In some embodiments, the amino acid residues in the SAPs can be naturally occurring or non-naturally occurring amino acid residues. Naturally occurring amino acids can include amino acid residues encoded by the standard genetic code as well as non-standard amino acids (e.g., amino acids having the D-configuration instead of the L-configuration or combinations of D- and L-amino acids), as well as those amino acids that can be formed by modifications of standard amino acids (e.g., pyrolysine or selenocysteine). Suitable non-naturally occurring amino acids include, but are not limited to, D-alloisoleucine(2R,3S)-2-amino-3-methylpentanoic acid, L-cyclopentyl glycine (S)-2-amino-2-cyclopentyl acetic acid. In other embodiments, another class of materials that can self-assemble are peptidomimetics. Peptidomimetics, as used herein, refers to molecules which mimic peptide structure. Peptidomimetics have general features analogous to their parent structures, polypeptides, such as amphiphilicity. Examples of such peptidomimetic materials are described in Moore et al., Chem. Rev. 101(12), 3893-4012 (2001). The peptidomimetic materials can be classified into four categories: α-peptides, β-peptides, γ-peptides, and δ-peptides. Copolymers of these peptides can also be used. Examples of α-peptide peptidomimetics include, but are not limited to, N,N′-linked oligoureas, oligopyrrolinones, oxazolidin-2-ones, azatides and azapeptides. Examples of β-peptides include, but are not limited to, β-peptide foldamers, α-aminoxy acids, sulfur-containing β-peptide analogues, and hydrazino peptides. Examples of γ-peptides include, but are not limited to, γ-peptide foldamers, oligoureas, oligocarbamates, and phosphodiesters. Examples of δ-peptides include, but are not limited to, alkene-based δ-amino acids and carbopeptoids, such as pyranose-based carbopeptoids and furanose-based carbopeptoids.

In certain embodiments, the SAP is AC5, AC5-V, AC5-G or TK45 (also known as AC1) (Arch Therapeutics, Inc., see www.archtherapeutics.com).

Also relevant to the present application is pending US Patent Application Publication No. 2021/0299326. As disclosed in that application, useful effects were observed resulting from sterilization of SAPs in water using irradiation. The samples included PuraSinus, (Ac-RADARADARADARADA-NH₂, RADA16 (SEQ ID NO:1)) 2.5% in water, IEIK13 (Ac-IEIKIEIKIEIKI-NH₂ (SEQ ID NO:3)) 1.3% in water, and QLEL12 (Ac-QLELQLELQLEL-NH₂ (SEQ ID NO:4)) 0.15%. Each of these self-assembling peptides are capable of forming a hydrogel when applied to a biological tissue (e.g., in situ) at about neutral pH. Generally, the SAP concentration in water will range from about 1% to about 5% weight/volume although this range is not exclusive. As disclosed in US Patent Application Publication No. 2021/0299326, it was found that gamma-irradiation sterilization enhanced the rheological properties of certain self-assembling peptide solutions and hydrogels (i.e., RADA16 and IEIK13) without the anticipated noteworthy degradation, while certain other peptides showed the expected significant degradation and viscosity drop after gamma irradiation (i.e., QLEL12). As known in the art, relatively pure SAPs, “relatively pure” meaning 70% or more of a particular peptide of a given exact sequence in a preparation are full length, it is commercially non-feasible to synthesize and purify a substantial amount using conventional filter sterilization due to high peptide is losses in the process.

Other observed useful effects of sterilization of selected SAPs using irradiation are of further value in the present invention. In preferred embodiments, the composition and methods of the invention maintain or improve the desired biological property(ies) such as hemostatic, anti-adhesion, prevention of re-bleeding, anti-stenosis, tissue occlusion, storage modulus, viscosity, and tissue void filling.

In US Patent Application Publication No. 2016/0317607, titled “Purified Amphiphilic Peptide Compositions and Uses Thereof,” additional observations of the properties of PuraSinus (RADA16—referred to in that application as PuraStat) with major peptide content over 65-70% make clear advantages of over the prior art. With respect to the above-mentioned application, the declaration of Eun Seok Gil, Ph.D., dated Apr. 24, 2021, reiterates the advantageous properties of purified SAPs of the exact same sequence wherein at least 75% of the peptides are full length. The tested solutions with less than 65-70% pure peptides were not observed to self-assemble into gels when exposed to about neutral pH and would therefore not be useful here.

The transparency of the above SAP gels, as noted in the above application, is advantageous for visualization of the tissue covered by the gel in vivo and for observation of wound healing prior to expected completed resorption of the matrix at about 30 days. This benefit of transparency is noted in European Patent Application Pub. No. 2581097 assigned to Arch Therapeutics, Inc. although the disclosed wound dressing in that application is not suitable for endoscopic administration.

### Methods of the Invention

PuraSinus as used in this study is comprised of the peptide Ac-RADARADARADARADA-NH₂, RADA16-I (SEQ ID NO:1) and potentially fragments derived therefrom, about 2.5% in water. As a soluble and transparent gel that can be applied through an endoscopic catheter, the gel's ease of application lends itself to its use deep in the nasal cavity, at the surgical site post-surgery, demonstrates improved healing compared to a control group not treated with a healing agent post-surgery and to subjects treated with FLOSEAL (Baxter Healthcare).

Currently in Australia, under the Australia Register of Therapeutic Goods, PuraStat has been approved for use in exudative hemorrhage from blood vessels and parenchyma of solid organs, vascular anastomoses and small vessels and capillary vessels of the GI tract (ARTG entry 267486). The self-assembling peptides (SAPs) disclosed herein demonstrate improved results following this type of surgery in studies developed in compliance with International Standard ISO 10993-1, “Biological evaluation of medical devices” (2016). All staff and facilities conducted the study in compliance with WHO Good Laboratory Practice.

The invention provides a method of reducing negative sequelae following endoscopic nasal surgery in mammals, the method comprising applying an effective amount of a self-assembling peptide (SAP) solution, the application taking place to a site of tissue injured as a result of endoscopic nasal surgery, trauma and/or pathology in the sinuses and/or turbinates wherein said SAP is applied via a syringe with (an adapted) nozzle or via endoscopic catheter to the site, thereby the SAP solution forms a gel when coming in contact with blood or other bodily fluids, and induces rapid hemostasis and/or reduces of synechiae formation, with the proviso that RADA16 (SEQ ID NO:1) consisting of L-amino acids, is excluded from the SAPs being used in endoscopic turbinoplasty. In some embodiments, the SAP is chosen from the group consisting of: a) RADA16 (SEQ ID NO:1), b) KLD12 (SEQ ID NO: 2), c) IEIK13 (SEQ ID NO:3), and d) KLDL12 (SEQ ID NO:5). In some embodiments, the SAP solution contains an SAP which is at least: 65%; 70%; 75%; 80%; 85% pure with respect to the full-length peptide relative to its truncated forms. In some embodiments, the SAP comprises IEIK13 (SEQ ID NO:3) at a concentration of 0.5% and 5%, preferably, 0.5% to 3.0%. In some embodiments, endoscopic surgery is turbinoplasty, septoplasty, and/or sinus surgery. In some embodiments, the site of SAP solution application includes one or more sinus orifices, thereby preventing formation of “clogging” or blocking synechiae that may close the sinus air circulation and create an environment for chronic sinus infection. In some embodiments, the site of SAP solution application is located at the inferior of turbinate/septum, and/or the middle of turbinate/septum.

The outcomes of the methods can be assessed by several means, for example, by reduction of the synechiae formation by at least by 20%, 30%, 40%, 50%, 60%, 70%, 80% or more. In some embodiments, healing over a period of weeks after the endoscopic surgery improves in one of more the following observable metrics:
A. Reduction in epithelial erosion;
B. Reduction in inflammatory infiltrate;
C. Reduction in submucosal fibrosis;
D. Improved angiogenesis;
E. Presence/absence of seromucous glands; and
F. Presence/absence of goblet cells.

Other aspects of the invention would be apparent to those of skill in the art based on the present description, including the Examples and the appended claims.

### Example 1: SAP Hydrogels for Hemostasis and Prevention of Synechiae Formation Following Turbinoplasty in a Sheep Model of Chronic Rhinosinusitis

Summary—Thirty healthy sheep underwent endoscopic surgery to create uniform nasal mucosal injuries on the middle turbinate and opposing nasal septum as a model for chronic rhinosinusitis (CRS). The effects of endoscopic treatment of the surgical wounds with a commercially available version of a self-assembling peptide (RADA16-I (SEQ ID NO: 1) 2.5% in water; PuraSinus; 3dmatrix.com/us/products/purasinus/), were compared to results using treatment with a gelatin/human thrombin product (FLOSEAL) and also untreated controls. The method of treatment for the surgical sites (two sites/sides per sheep) were selected randomly by computer. Primary outcomes of intra-operative hemostasis were recorded, as provided in Appendix 1, with synechiae formation and wound healing evaluated endoscopically at 2 weeks under sedation and at 6 weeks via post-mortem examination in situ and by histological intra-operative hemostasis time improved with RADA16 and Gelatin-thrombin versus Control wounds (139.7±56.2 s, 145.4±58.1 s, and 224.0±69.9 s, respectively; p<0.0001 for both comparisons). Two-week synechiae scores (maximum 4 points) were similar in Controls (2.9±1.8 points) and Gelatin-thrombin (3.1±1.6) wounds (p>0.05), but were reduced in RADA16 sites by 91% versus Controls and 92% versus Gelatin-thrombin treatment (0.3±0.6 points; p<0.0001 for both comparisons). Six-week synechiae scores were similar in Control (1.1±1.7) and Gelatin-thrombin (1.7±2.0 points) wounds (p>0.05), but reduced 100% in RADA16-treated wounds. Synechiae occurred in fewer RADA16-treated sites at 2 weeks (20%) versus Gelatin-thrombin (80%) and Controls (75%; p<0.01) and at 6 weeks (0%, 50% and 35%, respectively; p<0.01). RADA16 was associated with significantly lower 6-week histopathology scores, driven by reduced submucosal fibrosis and angiogenesis. Additionally, PuraSinus was more commonly associated with enhanced mucosal regeneration as measured by epithelial erosion/regeneration, inflammatory infiltrate, submucosal fibrosis, angiogenesis, and sero-mucous gland/goblet cell regeneration on histopathological scores.

Details of the Sheep Study—Endoscopic nasal surgery targeting the turbinates and sinuses has become a cornerstone in managing chronic rhinosinusitis. A sheep model was chosen to test treatment of an endoscopic surgical wound deep in the sinuses with PuraSinus (RADA16-I; (SEQ ID NO:1) 2.5% in water) compared to controls as a model for rhinoplasty in humans both due to the size of the animals and the suitability of the sheep model for comparison with this kind of surgery in humans as described below. PuraSinus (ARTG: 267486; (SEQ ID NO:1)) was supplied by 3-D Matrix Europe SAS as a 2.5% weight/volume solution of synthetic SAPs in water having a pH of about 2.3. Upon contact with physiologic tissue and/or liquid like blood, the pH and/or ionic strength of PuraSinus (SEQ ID NO:1) increases, triggering the self-assembly of the peptides into nanofibers, forming a beta-sheet network similar to an extracellular matrix. At a macroscopic level, the viscous solution forms a hydrogel that seals the opened vessels resulting in hemostasis. PuraSinus (SEQ ID NO:1) is biocompatible and free from infectious agents and is resorbed over time although some residue may remain for ≥30 days. It is supplied sterile in a syringe and delivered through a nozzle supplied by the manufacturer. It is stored at 2-8° C. In this example, FLOSEAL (ARTG: 192294) was obtained from Baxter Healthcare Pty Ltd. The FLOSEAL kit consists of a bovine-derived gelatin matrix, a human derived thrombin component, applicator tips, and several mixing accessories. The mixing accessories include a syringe with an integral female Luer connector attached, a small bowl, and a 5 mL syringe with needle attached. The mixing accessories are included to facilitate the reconstitution and mixing of the thrombin into the gelatin matrix. Applicator tips are included to facilitate the delivery of FLOSEAL to the site to be treated. The gelatin matrix consists of crosslinked gelatin granules and is provided sterile and non-pyrogenic in a standard disposable syringe.

The Thrombin (Human) component in FLOSEAL is a sterile, non-pyrogenic, freeze-dried, vapor-heated and solvent detergent-treated powder preparation made from pooled human plasma. The sodium chloride solution is a sterile, non-pyrogenic solution. After the constitution of the lyophilized thrombin in sodium chloride solution, the resulting Thrombin solution contains 500 IU/mL Thrombin (Human). FLOSEAL is a combination of the gelatin matrix and the thrombin component.

Thrombin must be added to the gelatin matrix prior to use. FLOSEAL is biocompatible, but not necessarily free from infectious agents or allergens, and resorbed within 6 to 8 weeks, consistent with normal wound healing (Ref FLOSEAL IFU). FLOSEAL is used as a hemostat for surgical procedures refractory to ligature or conventional procedures. It is stored at room temperature 2-25° C.

Animal subjects—Thirty healthy, castrated, previously unused male Merino sheep (Ovisaries) of no particular age or body weight were selected for the studies. The use of a sheep model has been validated for studying the outcomes of endoscopic nasal surgery (C. L. Shaw, et al., Aust. J. Otolaryngol. 4(1): 23-26 (2001)) with extensive use across numerous literature references evaluating the efficacy of similar hemostatic adjuncts (e.g., Valentine R. et al., Otolaryngol. Clin. North A. 42(5): 813-28 (2009)). Alternative models such as New Zealand white rabbits have been explored in the past, though the difficulty in acquiring Pasteurella-free rabbits has a risk of potentially confounding results. The sheep is an ideal model as the internal anatomy (sinuses, inferior and middle turbinates) is analogous to that of humans, and the size of the nasal cavity allows easy access of surgical equipment and endoscopes for post-operative monitoring. Each sheep underwent general anesthesia for the entire surgical procedure to prevent any potential intraoperative discomfort and/or pain. In addition, pre-operative and post-operative analgesics were used and the sheep were monitored for pain and distress.

There are no available validated in vitro assays or computer-simulated models that can mimic the complexity of post-surgical adhesion formation. The studies were conducted at USYD University Veterinary Centre Camden, Australia, and conformed with all applicable standards for humane treatment and care of the animals.

Animal Management—The animals were housed at the USYD University Veterinary Centre Camden in accordance with their established standards of care. The animals were kept in an outdoors pasture with daily monitoring. Ear tags were used to identify animal number, test code and date of surgery.

The sheep were allocated an area in a specialized UVCC pasture for daily feeding. Potable water was provided through species appropriate water containers or delivered through an automatic watering system. There were no contaminants present in the feed and water that were expected to impact the results of this study. UVCC is an accredited veterinary teaching hospital, clinic, and research center. All research conducted on the premises is subject to ethics approval from the University of Sydney's Animal Ethics Committee as required by the NSW Animal Research Act (1985).

Associates involved in this study were appropriately qualified and trained with experience operating on large animals and in endoscopic nasal surgery. It was determined that the use of sedation, analgesia or anesthesia was necessary during the routine course of this procedure. All anesthetics, analgesic, and other medications were given or altered at the discretion of the veterinarian or anesthesiologist in accordance with standard veterinary practice and the study objectives. This applies to specific medication, dose, and dosing intervals. None of the animals became injured, ill, or moribund, so as to require care, such as euthanasia, prior to the end of the study.

Surgical Methods—On the day prior to surgery, the animals were weighed, and each nostril was randomly assigned by computer randomization to receive PuraSinus, FLOSEAL or no treatment to each nostril after the surgical turbinoplasty. Each animal had food withheld for 24 hours and water withheld for 8 to 12 hours prior to the day of surgery as per the standard anesthetic procedure. The animals were sedated with a weight adjusted dose of intravenous diazepam. Following sedation, a percutaneous jugular catheter was inserted and secured, with induction achieved by further weight adjusted doses of intravenous diazepam and ketamine. The animals were placed in sternal recumbency for insertion of a size-appropriate endotracheal tube. Anesthesia was maintained through inhaled 1-2% isofluorane with parenteral propofol and ketamine given when necessary. Each animal was then given a prophylactic dose of long-acting antibiotic, weight adjusted oxytetracycline to cover for postoperative infections. The nasal cavity was prepared by packing with surgical pledgets soaked in 1% lignocaine and phenylephrine mixture. The middle turbinate was infiltrated with the same lignocaine/phenylephrine mixture via a small gauge needle in numerous sites over the middle turbinate. The use of vasoconstrictors and decongestants such as lignocaine and phenylephrine both topically and injected locally is standard pre-operatively in patients undergoing turbinoplasty (Ahmed S. et al., J. Coll. Physicians Surg. Pak. 26(6): 531-2 (2016)) The animals' vital signs were monitored throughout the procedure.

Prior to making incisions, all sheep were first examined under endoscope to screen for any nasal septum deviations. Only animals that had straight septum and uniform space between septum and middle turbinate were used.

Operative procedure—A uniform sized 2 cm×3 cm full thickness mucosal lesion with removal of the periosteum was created on the lower edge of the medial aspect of the middle turbinate using a Medline Microdebrider. The large size of the vomer in the sheep serves as an anatomical landmark to standardize the position of the lesion.

Additionally, a uniform sized 2 cm×3 cm mucosal lesion was created contralaterally on the septum. Due to the volumetric size of the middle turbinate in sheep, the creation of a wound in close proximity to a contralateral septal wound allowed us to create an environment prone to adhesions so that we could best study the adhesion-preventing properties of the various products. The surgeon was not made aware of the product intended for the wound prior to its creation.

Depending on the results of the randomization, either PuraSinus (SEQ ID NO:1) 2.5% w/v in water, FLOSEAL or nothing was applied to the lesion immediately following its creation. FLOSEAL was prepared intraoperatively by mixing the Thrombin liquid and microbeads as per manufacturer's instructions. PuraSinus (SEQ ID NO:1) was applied in its supplied form without any preparation as per manufacturer's instructions. Both products were applied via a 20 cm long applicator in a posterior to inferior fashion along the length of both lesions within the nostril. The surgeon was not made aware of which product was to be applied to which wound until they were handed the allocated product. Further blinding as to the specific product used was not possible, as the physical appearance of PuraSinus (SEQ ID NO:1) and FLOSEAL are distinctly different with different methods of application. Five milliliters (ml) of either product were applied uniformly over the wound as per the product's intended method of application. In the case of PuraSinus (SEQ ID NO:1), this was achieved through uniform application in direct supposition with the bleeding edge. In order to best assess the performance of the product in isolation, no other hemostatic adjuncts such as electrocautery or nasal packing were used.

Intraoperative observations—The degree of bleeding/hemostasis was assessed by an independent observer who was blinded to the appearance of FLOSEAL and PuraSinus (SEQ ID NO:1), and to the background of these two hemostatic agents. The surgical field is graded by the time taken in seconds to achieve complete hemostasis with no additional blood seen to be draining from the nose. Observed times to hemostasis for each site are reported in Appendix 1. Any suctioning was performed through careful introduction of a narrow suction catheter tip into the empty space between the septum and the application area in a manner that did not agitate the product.

Anesthetics recovery—Postoperatively, animals were moved to a recovery area with continued monitoring of vital signs. Sternal recumbency was maintained to allow for ruminal gas to escape. Extubation was attempted once the animals were able to swallow and chew spontaneously. Once recovered, they were given standard postoperative analgesia through a combination of opioids and NSAIDs, and standard postoperative antibiotics. Locally acting analgesia was avoided so as to minimally interfere with the operative site.

Post-operative observations—Post recovery from anesthesia, the animals were returned to their pens with ongoing daily monitoring for 14 days, including bloody nasal discharge, temperature, and food and water intake. On each postoperative day, the animals were examined externally by an independent observer, with no involvement in the operation, for the presence of bloody nasal discharges. External blood was cleaned off after each examination followed with irrigation of each nostril using 20 ml of saline solution. Regular irrigation of the nose and sinuses is standard in post-operative care following nasal surgery.

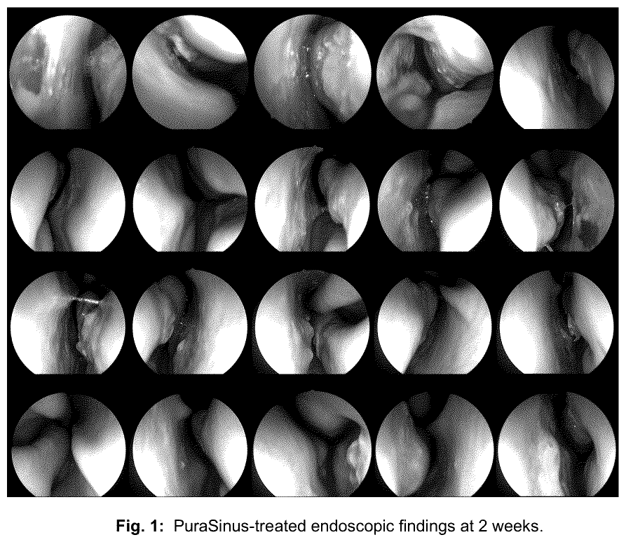

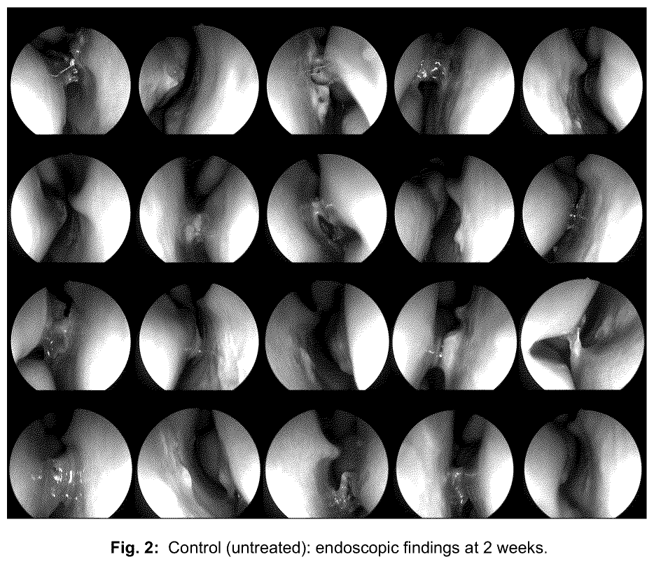

The sheep were sedated and examined via endoscope again on day 14 for the presence of adhesions and graded by an independent observer using the grading scheme below for sheep nasal adhesions. Representative endoscopic images for sites (two sites/sides per sheep) treated with PuraSinus (SEQ ID NO:1) are shown in FIG. 1 and untreated control sites are shown in FIG. 2, respectively.

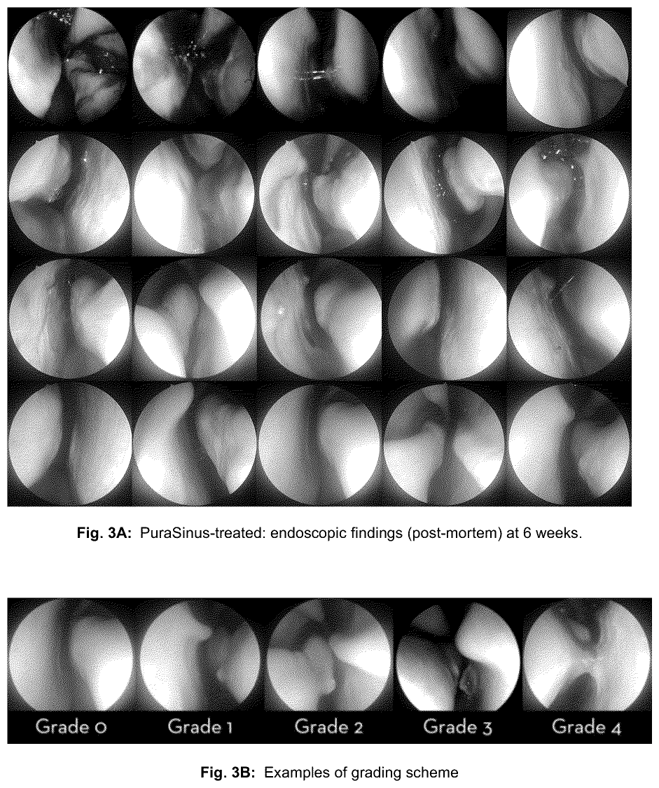

The sheep were euthanized at day 42, with a necropsy performed by the same independent observer grading intranasal adhesion formation and extension. Representative in situ endoscopic images for sites treated with PuraSinus (SEQ ID NO:1) are shown in FIG. 3A. The observations were classified with respect to degree of adhesion formation as seen by endoscopic imaging both on day 14 and day 42 according to the scheme laid out in Table 2 below. Example endoscopic images corresponding to each grade are shown in FIG. 3B.

TABLE 2

| Grade | Classification |
|---|---|
| 0 | No intranasal adhesions/synechiae |
| 1 | Intranasal adhesion extends less than 25% to septum |
| 2 | Intranasal adhesion extends 25-50% to septum |
| 3 | Intranasal adhesion extends more than 50% to septum |
| 4 | Completely formed adhesion/synechiae extending to septum |

Termination—At day 42, the animals were euthanized with standard terminal procedures through an intravenous injection of sodium pentobarbital. Post-termination, the mucosa at the operated middle turbinate sites were excised and prepared via hematoxylin and eosin staining by a veterinary histopathologist using 6 different markers of wound healing as shown in Table 3.

TABLE 3
Histological indicators of wound healing

| | |
|---|---|
| 0-3 | Epithelial erosion |
| 0-3 | Inflammatory infiltrate |
| 0-3 | Submucosal fibrosis |
| 0-3 | Angiogenesis |
| −1-1 | Sero-mucous glands |
| −1-1 | Goblet Cells |

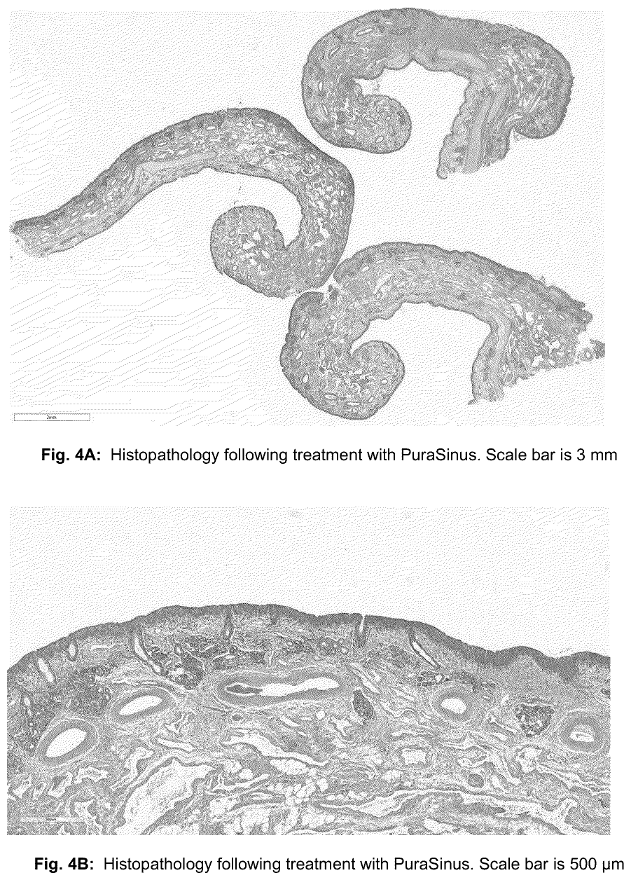

Representative tissue sample images taken from the surgical wound site after 6 weeks post-surgery are shown in FIGS. 4A and 4B for sites treated with PuraSinus. Images of tissue taken from tissue treated with FLOSEAL are shown in FIGS. 5A and 5B. Observations by blinded observers of specimens from each of the sheep graded according to the factors listed in Table 2 are given in Appendix 3 and summarized below. Those results suggest an improved outcome for treatment with PuraSinus compared to treatment with FLOSEAL.

Statistical analysis—Data are presented as mean±SD, mean±95% confidence intervals (CIs), or percentages, as indicated. Continuous variables were compared across the three groups using one-way ANOVA followed by Bonferroni's post-test to adjust for multiple corrections. Categorical data were evaluated using Fisher exact test. A two-tailed p-value<0.05 was considered statistically significant. Prism v.5.03 statistical and graphing software (GraphPad Software Inc., San Diego, CA) was used. Observed individual results are given in Appendix 3.

Sample size calculation—In a previous study (J. G. Medina and S. Das, Laryngoscope 123(1): 42-7 (2013)), it was found that the control group (14 sites) had an 86% adhesion rate, with a 95% CI (confidence interval) of ±17.5, and the treatment group (10 sites, but using a chitosan hemostatic agent) had a 10% adhesion rate with a 95% CI of ±16.5. The mean difference between the groups was 76% with 95% CI of ±34. The mean difference was robust as all values of its 95% CI are positive—meaning the probability of treatment group having a lower adhesion rate than control group is very high. However, the margin of error was quite large (±34), therefore making it difficult to judge clinical significance. We calculated that 20 surgical sites (10 animals) in each group should give a confidence interval of ±20%.

Detailed sheep model observations—Uniform middle turbinate injuries were successfully created across all 30 sheep between 2018 and 2019 through four groups of 4, 9, 9 and 8 sheep respectively. The large size of the nasal cavity and nasal vomer allowed us to readily re-create standardized injuries in all the sheep both in terms of size and location. 4 ml of product total was applied to the two sites allowing for adequate coverage of the injury.

After the initial operative procedure in the pilot group, operative sedation rather than general anesthesia was used for the day 14 follow-up. This option was both safer for the animals and proved to be more cost-efficient. All sheep survived to the day 42 mark and proceeded to euthanasia and subsequent necropsy and histopathology.

Clinical observations—Effective compliance to routine nasal douching in the post-operative setting was enforced through daily irrigation with 20 ml saline in the first 14 days. No excessive external bleeding was reported with daily observations of the sheep.

The results presented in Appendix 2 concerning observed synechiae formation can be summarized as follows:

At two weeks post-surgery the number of sites observed for each grade in each group as defined in Table 2, was as follows:

| | FLOSEAL | PuraSinus | Control |
|---|---|---|---|
| Grade 0 | 4 | 16 | 5 |
| Grade 1 | 0 | 3 | 1 |
| Grade 2 | 1 | 1 | 0 |
| Grade 3 | 1 | 0 | 0 |
| Grade 4 | 14 | 0 | 14 |

The best results, meaning lowest observed synechiae formation, were for surgical sites treated with PuraSinus (SEQ ID NO:1). Six weeks post-surgery the observed results concerning degree of synechiae formation also show a statistically significant improvement over both untreated controls and wounds treated with FLOSEAL as follows:

| | FLOSEAL | PuraSinus | Control |
|---|---|---|---|
| Grade 0 | 10 | 20 | 13 |
| Grade 1 | 2 | 0 | 2 |
| Grade 2 | 0 | 0 | 0 |
| Grade 3 | 0 | 0 | 0 |
| Grade 4 | 8 | 0 | 5 |

These results demonstrate that PuraSinus (SEQ ID NO:1) was more effective in inhibiting the formation of synechiae between surgically traumatized tissue and nearby post-nasal tissue relative to both FLOSEAL and untreated controls.

Histopathology observations—Vertical incisions through the roof and floor of the nasal cavity were performed during necropsy after endoscopic evaluation. This allowed both lateral nasal walls to be opened outwards from the septum whereby the operated mucosa adjacent to the vomer could be easily identified and excised atraumatically.

Specimens were preserved in 10% neutral buffered formalin and transported for hematoxylin and eosin staining and slide preparation. Example specimens are shown in FIGS. 4A and 4B for PuraSinus treated tissue and FIGS. 5A and 5B for FLOSEAL treated tissue. Individual sample observations are presented in Appendix 3. Those results can be summarized as follows:

Epithelial erosion (EE): Number of sites by grade

| | FLOSEAL | PuraSinus | Control |
|---|---|---|---|
| Grade 0 | 4 | 5 | 7 |
| Grade 1 | 4 | 8 | 3 |
| Grade 2 | 6 | 6 | 5 |
| Grade 3 | 6 | 1 | 5 |
| Mean score | 1.75 | 1.15 | 1.35 |

Inflammatory infiltrate (II):

| | FLOSEAL | PuraSinus | Control |
|---|---|---|---|
| Grade 0 | 1 | 3 | 0 |
| Grade 1 | 12 | 13 | 13 |
| Grade 2 | 7 | 4 | 6 |
| Grade 3 | 0 | 0 | 1 |
| Mean score | 1.30 | 1.05 | 1.35 |

Submucosal fibrosis (SF):

| | FLOSEAL | PuraSinus | Control |
|---|---|---|---|
| Grade 0 | 0 | 4 | 0 |
| Grade 1 | 3 | 12 | 4 |
| Grade 2 | 3 | 3 | 6 |
| Grade 3 | 14 | 1 | 10 |
| Mean score | 2.55 | 1.05 | 2.3 |

Angiogenesis (A):

| | FLOSEAL | PuraSinus | Control |
|---|---|---|---|
| Grade 0 | 3 | 9 | 3 |
| Grade 1 | 4 | 6 | 6 |
| Grade 2 | 6 | 4 | 7 |
| Grade 3 | 7 | 1 | 4 |
| Mean score | 1.85 | 0.85 | 1.60 |

Sero-mucous glands (SMC):

| | FLOSEAL | PuraSinus | Control |
|---|---|---|---|
| Grade −1 | 13 | 6 | 12 |
| Grade 0 | 6 | 14 | 8 |
| Grade +1 | 1 | 0 | 0 |
| Mean score | −0.6 | −0.3 | −0.6 |

Goblet cells (GC):

| | FLOSEAL | PuraSinus | Control |
|---|---|---|---|
| Grade −1 | 7 | 8 | 6 |
| Grade 0 | 12 | 6 | 1 |
| Grade +1 | 1 | 6 | 13 |
| Mean score | −0.3 | −0.1 | −0.25 |

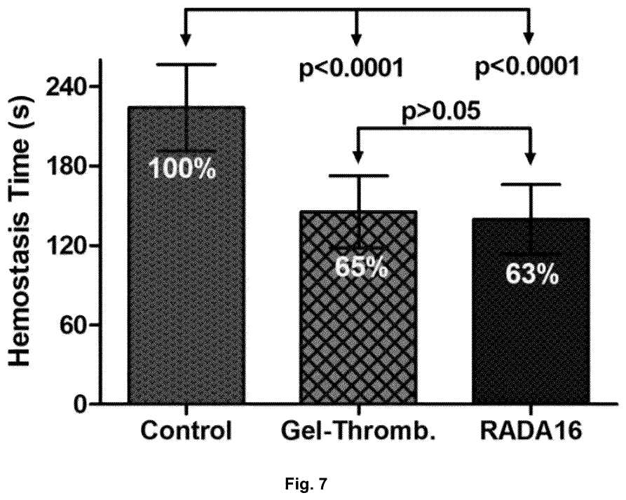

Summary of Results—The subject article PuraSinus as well as the product FLOSEAL demonstrate a statistically significant reduction in time to hemostasis as compared to the untreated control group. PuraSinus was associated with a statistically significant lower incidence of adhesions at both 2 and 6 weeks as compared to both the FLOSEAL and untreated control groups—see FIG. 7.

Histopathology results suggest favorable outcomes for wound healing enhancement across all 6 metrics (epithelial erosion, inflammatory infiltrate, submucosal fibrosis, angiogenesis, sero-mucous glands, and Goblet cells) in the PuraSinus group compared to the FLOSEAL group; also see FIGS. 4A, 4B, 5A, and 5B.

Records—All raw data, photographs on digital media, wet tissues, blocks, and tissue slides pertaining to this study and a copy of the final report are retained in the coordinator's archives in accordance with WHO Good Laboratory Practice and USYD Ethics.

### Example 2: Use of SAPs in Functional Endoscopic Sinus Surgery for Hemostasis, Wound Healing and Reduction of Adhesion Formation

Summary—In a retrospective clinical study of 94 adult human patients undergoing a variety of Functional Endoscopic Sinus Surgeries (FESS), including some patients who underwent turbinoplasty and/or septoplasty indicated for chronic rhinosinusitis (CRS), intra-operative application of RADA16-I SAP (PuraSinus; (SEQ ID NO:1) 2.5% in water) to the surgical wound resulted in effective hemostasis in the absence of other hemostatic agents and devices in most patients. The absence of adhesions, also known as synechiae, was reported in almost 90% of these cases at the second of two follow-up examinations for each of the patients. This study involved human patients with a more varied range of characteristics and sinonasal pathologies than other studies and demonstrates the utility of SAPs in inducing hemostasis, promoting wound healing, prevention of adhesions during recovery longer term (the incidence of adhesions generally increases over time post-surgery), and absence of pain and patient discomfort during recovery. Also, no revision surgeries were indicated in this study.

Introduction—Functional endoscopic sinus surgery (FESS), including turbinoplasty, is one of the most common surgeries performed in otolaryngology for patients suffering from chronic rhinosinusitis (CRS) and failing maximal medical therapies (J. Selvarajah et al., Int. J. Mol. Sci. 21(2):480 (2020)). CRS affects millions of individuals and has a significant impact on quality of life (M. S. Benninger et al., Otolaryngology-Head and Neck Surgery. 129(3 Suppl): S1-32 (2003)). In patients with failed maximal medical therapy, suspected anatomical abnormalities or complicated CRS who cannot be managed conservatively, surgical intervention is warranted (K. C. Welch and J. A. Stankiewicz, The Laryngoscope 119(11):2258-68 (2009)). The surgical approach aims to restore drainage and airflow throughout the affected sinuses (R. K. Weber and W. Hosemann, GMS current topics in otorhinolaryngology, head and neck surgery; 14 (2015)).

Given the vascularity of nasal mucosa, sinonasal surgery carries a moderate bleeding risk particularly in inflammatory conditions such as CRS (Pant H., Otolaryngologic Clinics of North America. 49(3):655-76 (2016)). As previously explained, hemostasis is necessary to prevent postoperative complications, such as delayed bleeding, poor wound healing and synechiae formation. These SAPs in aqueous solution were used to treat a series of consecutive human patients undergoing FESS. The aim of this study was to assess the effectiveness of PuraSinus in achieving rapid intraoperative and post-operative hemostasis and in reducing adhesions following FESS.

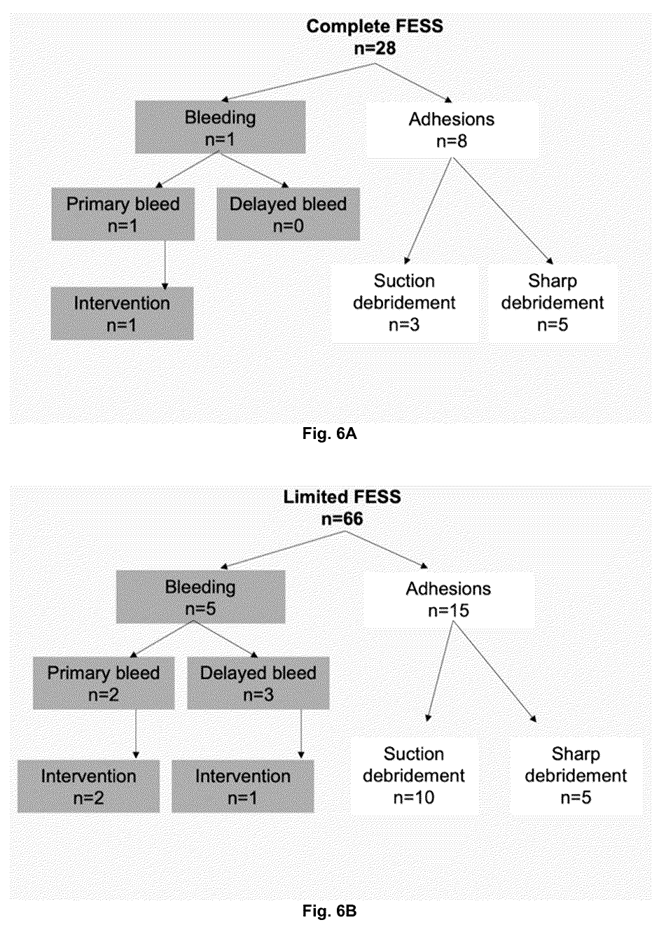

Study design—This study was a retrospective chart review of 94 consecutive human patients who underwent FESS and who were operated on by a single surgeon in a single center in Perth, Western Australia. FIGS. 6A and 6B show a flowchart of patients based on extent of surgery and outcomes. Only patients treated with PuraSinus alone (no other hemostatic agent was used) were included in the analysis. Ethics approval was obtained from St John of Good Health Care Human Research Ethics Committee.

Patient population—The study included adult human patients who underwent FESS procedures and also included patients who underwent FESS in combination with nasal surgery (septoplasty and/or turbinoplasty). Both primary and revision cases were included. Patients were discharged the day after surgery with a recommendation to perform saline nasal irrigations no less than four times a day. Patient characteristics, indication for surgery, extent of surgical intervention, and bleeding and adhesion information was recorded.

Surgical categories—Procedures were performed according to the minimally invasive sinus technique (MIST), including combination of maxillary antrostomy, ethmoidectomies, sphenoidotomies and frontal sinus (Draf Ila, or Draf 111), turbinate reduction and/or septoplasty. FESS procedures were categorized into two groups: complete FESS (including all sinuses—maxillary antrostomy, ethmoidectomy, sphenoidotomy, and frontal sinusotomy) or limited FESS (anything less than complete FESS, usually involves antrostomy and ethmoidectomy). The surgical extent was dictated by the patient's condition and the surgeon's clinical judgement.

Material used—PuraSinus (licensed as PuraStat in Australia, 3D-Matrix)/RADA16-I (SEQ ID NO:1) 2.5% w/v in water is a transparent SAP solution supplied sterile in a pre-filled syringe (available in a 3 mL and 5 mL form) ready for use with a thin application nozzle suited for endoscopic catheter administration. The syringe is stored at between 2° C. and 8° C. At completion of the FESS procedure, PuraSinus was applied to the surgical wound in a thin and even layer as close as possible to the bleeding point. A similar application technique has been outlined previously (M. Lee et al., Int. J. Surg. Case Rep. 41: 461-4 (2017)). Gravity was sometimes used to reach the posterior part of the inferior turbinate. Tissues treated with the hydrogel were visible via endoscope given the gel's transparency.

Outcomes and objectives—The objectives of this study were to evaluate the effectiveness of PuraSinus in rapidly achieving intraoperative hemostasis and to assess the incidence of delayed bleeding (24 hours postoperatively) and synechiae formation via endoscopic observation of the nasal mucosa at follow-up visits. Primary bleeding was defined as bleeding occurring within 24 hours of surgery.

Data analysis—Statistical analysis was performed using descriptive statistics for continuous variables and patient demographics were reported. The rate of successful hemostasis and incidence of synechiae formation were calculated as a proportion of total patients undergoing FESS.

Detailed Results—Results were analyzed from a total of 94 human patients who underwent sinus surgery from May 2017 to February 2021. Thirty-eight males and 56 females, with ages ranging from 18 to 83 years (median 48 years, mean±SD 47.9±15.8 years) were included in the study. Patient characteristics are summarized in Table 4. The most common indication for undergoing surgery was chronic rhinosinusitis (CRS) with or without nasal polyposis. Twenty-eight patients underwent a complete FESS procedure, of which majority also underwent a septoplasty (25/28) and turbinate reduction surgery (27/28). There were 66 patients who had a limited FESS procedure, of which a majority also underwent a septoplasty (59/66) and turbinate reduction surgery (62/66). Of the total 94 patients, seven were considered revision surgery. These flow-chart designs are illustrated in FIGS. 10A and 10B.

TABLE 4
Patient characteristics

| CHARACTERISTICS | PATIENTS (n = 94) | |
|---|---|---|
| Age, mean years (range) | 48 | (18-83) |
| Sex, n (%) | | |
| Female | 56 | (59.6%) |
| Male | 38 | (40.4%) |
| Surgical indication, n (%) | | |
| ARS or Recurrent ARS | 13 | (13.8%) |
| CRSSNP | 37 | (39.4%) |
| CRSwNP | 41 | (43.6%) |
| CSF rhinorrhea | 1 | (1.1%) |
| Sinonasal tumour | 2 | (2.1%) |
| Surgical extent, n (%) | | |
| Complete FESS | 28 | (29.8%) |
| Limited FESS | 66 | (70.2%) |
| Primary or revision FESS, n (%) | | |
| Primary FESS | 87 | (92.6%) |
| Revision FESS | 7 | (7.4%) |
| Adjuvant surgical procedures, n (%) | | |
| Septoplasty | 84 | (89.4%) |
| Turbinate reduction | 89 | (94.7%) |
| Polypectomy | 47 | (50.0%) |
| First post-operative review, mean days (range) | 13.9 | (7-30) |

ARS: Acute Rhinosinusitis;
CRSsNP: Chronic Rhinosinusitis without Nasal Polyps;
CRSwNP: Chronic RhinoSinusitis with Nasal Polyps;
CSF: CerebroSpinal Fluid

Postoperative Bleeding—A total of six patients (6.4%) experienced postoperative bleeding. These results are summarized in Table 5. One of the six patients had undergone a complete FESS and the other five had a limited FESS. There were three patients with postoperative bleeding within 24 hours after surgery, one of whom may have had a preexisting bleeding disorder. All three of these patients required cauterization of the surgical wound for hemostasis. In addition to cautery, Surgiflo (absorbable gel hemostat) was used in one patient, and a second application of PuraSinus was successfully used in another. Of the three patients who experienced delayed bleeding postoperatively (i.e., more than 24 hours after surgery), only one with a previously diagnosed bleeding disorder required intervention. The other two patients with secondary bleeds postoperatively were managed conservatively. In total, four patients (three primary bleeds and one secondary bleed) all of whom had also undergone a septoplasty and turbinate reduction surgery in combination with their limited FESS (3 patients) or complete FESS (1 patient), required intervention for bleeding (4/94: 4.3%). Thus, the application of PuraSinus (RADA16-I; (SEQ ID NO:1) 2.5% in water) in achieving hemostasis in this study was 95.7% (90/94). There were no re-bleeds recorded from any of the patients with primary or secondary bleeds.

TABLE 5
Results for patients with postoperative bleeding

| CHARACTERISTICS | PATIENTS (n = 94) |
|---|---|
| Bleed | 6 (6.4%) |
| Primary | 3 (3.2%) |
| Secondary | 3 (3.2%) |
| Gender | |
| Female | 5 |
| Male | 1 |
| Extent of surgery | |
| Complete FESS | 1 |
| Limited FESS | 5 |
| Intervention required | 4 (4.3%) |
| Primary bleed | 3 (3.2%) |
| Secondary bleed | 1 (1.1%) |
| Type of intervention | |
| Cautery + Surgiflo | 1 |
| Cautery + additional PuraSinus | 1 |
| Cautery alone | 1 |
| Packing | 1 |
| Other factors | |
| Suspected or confirmed coagulopathy | 2 |
| On anticoagulation/antiplatelets post-op | 1 |
| Hemostatic effectiveness | 90 (95.7%) |

Adhesions—Nasal endoscopic examination of each patient was performed at a first follow-up visit occurring between 7 and 30 days after surgery (median 15 days, mean±SD 14.0±3.7 days). A subsequent follow-up examination of each patient was performed on average on day 42, ranging from 28 to 79 days post-surgery. No residual PuraSinus (SEQ ID NO:1) hydrogel was noted, and all patients expressed comfort with no pain at the surgical site. A total of 23 patients (24.5%) were found to have synechiae formation during the full follow-up period, eight of whom had undergone a complete FESS and 15 a limited FESS. Only 18 of these 23 patients were found to have synechiae at the first follow-up visit. The most common site found to have adhesion formation was the inferior turbinate/septum (n=11/23). Table 6 summarizes locations and characteristics of synechiae found in patients undergoing FESS. Debridement of the observed synechiae was performed in these cases in the outpatient setting by either suction (n=13) or scissors (n=10). Subsequently, at the second follow-up examinations 84 patients in total were found to have no adhesions (n=71) or adhesions that were easily removed via suction division (n=13). Thus, the desired outcome of intraoperative treatment with PuraSinus (SEQ ID NO:1) in achieving recovery without synechiae formation in these patients was nearly 90% (n=84/94). None of the patients who had required additional intervention for hemostasis subsequently presented with adhesion formation. Furthermore, no patients in this case series required revision surgery.

TABLE 6
Results for patients with adhesion formation

| | PATIENTS |
|---|---|
| CHARACTERISTICS | (n = 23/94) |
| Adhesion formation | |
| Total | 23 |
| At first follow up visit | 18 |
| Gender | |
| Female | 16 |
| Male | 7 |
| Extent of surgery | |
| Complete FESS | 8 |
| Limited FESS | 15 |
| Intervention (debridement) | |
| Suction | 13 |
| Scissors (sharp) | 10 |
| Adhesion location | |
| Inferior turbinate/septum | 12 |
| Medial turbinate/lateral wall | 7 |
| Inferior turbinate/septum + medial | 4 |
| turbinate/lateral wall | |
| Adhesion final outcome | |
| Resolved adhesions | 18 |
| Persisting adhesions | 5 |
| Patients without adhesions | 84/94 |

Discussion—We demonstrated that the application of PuraSinus (RADA16-I; (SEQ ID NO:1)), a peptide hydrogel, is effective in limiting bleeding and adhesions, while avoiding nasal packing post-FESS in most patients in this study. Up to 25% of postoperative hemorrhage in FESS may occur within 24 hours. Although a significant bleed can occur up to six weeks postoperatively, the most common time frame is between one and two weeks after FESS (Pant H. Otolaryngologic Clinics of North America. 2016; 49(3): 655-76). Similarly, in our study, of the six patients who experienced a bleed, three had postoperative bleeds on days two, five, and nine. The other half of the patients had a recorded primary bleed (occurring within 24 hours). This discrepancy is likely due to smaller numbers in our cohort, and contributing to this, was the fact that one of the patients with a primary bleed had a suspected coagulopathy. Unfortunately, there are varied definitions in the literature when investigating bleeding during and after FESS. For example, some studies have identified bleeding postoperatively in FESS as severe hemorrhage—a patient requiring a blood transfusion or a return to surgery for hemostasis or have recorded bleeding as a complication only when it necessitates a readmission (S. Suzuki et al., The Laryngoscope. 2015; 125(8):1785-91; V. Siedek et al., European Archives of Oto-Rhino-Laryngology. 2013; 270(1):141-8; Y-P. Wang et al., Journal of the Chinese Medical Association. 2011; 74(1):16-21; H. Khoury et al., The Laryngoscope. 2021; 131(5):E1422-E8). Excessive peri-operative bleeding has been reported to occur in approximately 5% of patients after FESS, with 0.8% of major bleeds needing a transfusion (C. Hopkins et al., The Laryngoscope. 2006; 116(8): 1494-9; J L. Antisdel et al., The Laryngoscope. 2016; 126: S5-S13). Comparably, in our study the rate of patients with any bleeding was 6.4% but the rate of those that required additional treatment was only 4.2% (n=4/94). None of the patients in this study required a blood transfusion. In a large retrospective study of 3,402 patients over 25 years, Stankiewicz et al. (2011) only considered bleeding as a complication when it necessitated packing or surgery for control (JA Stankiewicz et al., The Laryngoscope. 2011; 121(12):2684-701). Thus, according to our definition of hemostatic effectiveness, like that of Stankiewicz et al., 95.7% of patients (n=90/94) who underwent FESS did not have any bleeding postoperatively or, those that did, did not require any intervention.

The rates in our study are similar and are at the lower range of incidences previously stated (R. Valentine et al., American Journal of Rhinology & Allergy. 2010; 24(1):70-5; J L Antisdel et al., American Journal of Rhinology & Allergy. 2011; 25(4):268-71). A total 24.5% of patients were found to have adhesions throughout their postoperative follow-up appointment. However, only ten of those required sharp debridement, while the remaining 13 were easily separated with suction debridement. As a result, in this study 89.4% (n=84/94) of patients were free of adhesions at the second follow-up examination, where 71 patients had no adhesions found during the first follow-up period and 13 of these patients had adhesions easily removed. The majority of patients who had adhesions were found to be resolved after intervention. No patients in this study required revision surgery.

The incidence of adhesion formation and bleeding after sinus surgery differs considerably in the literature, and is likely related to the heterogeneity of studies, with confounding variables including the extent of surgery, septoplasty or other adjuvant surgery involved, patient factors (smoking and comorbidities), and the type of nasal packing used. Wang et al. (2015) conducted a systematic review and meta-analysis to compare the efficacy of non-absorbable vs absorbable nasal packing in FESS (T-C. Wang et al., European Archives of Oto-Rhino-Laryngology. 2015; 272(8):1825-31). Their study found that although there may be evidence that absorbable nasal packing can provide some improved outcomes when compared to non-absorbable packing after FESS, there was a lack of homogeneity between studies (Id.). They were unable to make definitive conclusions from their review and suggested the need for more randomized clinical trials on the topic. Similar findings were established in another meta-analysis and systematic review on middle meatal packing after FESS, where Hobson et al. (2015) concluded that it does not significantly decrease the risk of adhesion formation (CE. Hobson et al., American Journal of Rhinology & Allergy. 2015; 2 9(2):135-40).

PuraSinus (RADA16-I; (SEQ ID NO:1)) 2.5% w/v in water, as a topical hemostat, has been used extensively in gastrointestinal endoscopic procedures (G. de Nucci et al., Endoscopy. 2020; 52(09):773-9; M. Pioche et al., Endoscopy International Open. 2016; 4(4):E415; S. Subramaniam et al., Endoscopy. 2021; 53(01):27-35; S. Subramaniam et al., United European Gastroenterology Journal. 2019; 7(1):155-62; T. Uraoka et al., Gastrointestinal Endoscopy. 2016; 83(6):1259-64), as well as in cardiovascular surgery (S. Giritharan et al., Journal of Cardiothoracic Surgery. 2018; 13(1):1-7; H. Masuhara et al., Annals of Thoracic and Cardiovascular Surgery. 2012:oa. 12.01977; M. Morshuis et al., The Journal of Heart and Lung Transplantation. 2019; 38(4):5194), with successful hemostasis rates ranging from 72.6-100% (S. Sankar et al., Frontiers in Bioengineering and Biotechnology. 2021; 9:465). However, clinical evidence within the field of ENT surgery is limited. Lee, et al. (2017) published a case series of 60 patients undergoing turbinate reduction surgery (TRS/Turbinoplasty) (Lee M. et al., International Journal of Surgery: Case Reports. 2017; 41:461-4). Comparable to this current study, exclusion criteria included any concurrent hemostatic adjuncts, and a similar technique of PuraSinus (called PuraStat in that paper) application and standard postoperative instructions were used. Lee et al., (2017) found that PuraSinus was very effective, with none of their patients experiencing bleeding or adhesion formation postoperatively. Although this an excellent outcome, it does differ from the total adhesion and bleeding rates in our study—24.5% and 6.4%, respectively. This is likely due to several differences between the two studies. Lee et al.'s case series includes patients with TRS/Turbinoplasty surgery only, whereas the present study examines a cohort of patients who have undergone FESS (complete or limited) including TRS, septoplasty and polypectomies. As this involves a greater extent of disease and surgical intervention, there may have been an increased risk of adhesions and bleeding (Stankiewicz JA. et al., The Laryngoscope. 2011; 121(12):2684-701). Additionally, data was recorded only from the first follow-up at four weeks in the case series of 60 patients, unlike this current study where a longer follow-up extended up to several months, resulting in identification of a further five patients with adhesions (whereas only 18 of 23 patients were identified at the first follow-up appoinent). It was noted that all patients were compliant with their postoperative care and nasal irrigation in the case series by Lee et al. (2017). Nasal saline irrigation is almost universally recommended post-FESS and plays an important role in wound healing and prevention of crusting and synechia formation (Lee et al. Archives of Otolaryngology—Head & Neck Surgery. 2007; 133(8):776-9; Shoman et al., Journal of Otolaryngology—Head & Neck Surgery=Le Journal d'oto-rhino-laryngologie et de chirurgie cervico-faciale. 2009; 38(1):112-8; Yoo F. et al., International Forum of Allergy & Rhinology. 2018; 8(1):32-40). Patient compliance with post-operative care was not formally recorded in our study and could impact patient outcomes. Despite these differences, we found a promising overall effectiveness of PuraSinus for hemostasis (95.7%) and and for prevention of adhesion formation (89.4%).

This study has several limitations. There was no control group, resulting in difficulty making comparisons to other types of hemostatic agents or packing. Although all surgeries were performed by a single surgeon in a single center, the patient population is heterogenous, including extent of disease and surgery, surgical indication, and adjuvant operations performed. Also, certain variables that may influence outcome were not recorded, such as associated comorbidities, smoking history, and patient compliance (as aforementioned). Furthermore, it would be valuable to identify the site of postoperative bleeds, as was done with postoperative adhesions. Quantifiable information to assess outcomes can be obtained, for example, by using grading of severity using the scarring component of the Lund-Kennedy endoscopic score (OA Henriquez et al., The Laryngoscope. 2013; 123(11):2615-9).

However, these results demonstrated that the relatively simple technique of PuraSinus application in patients undergoing sinus surgery was effective in achieving hemostasis, reducing adhesion formation, and avoiding nasal packing in most patients.

While this invention has been particularly shown and described with references to example embodiments thereof, it will be understood by those skilled in the art that various changes in form and details may be made therein without departing from the scope of the invention encompassed by the appended claims.

APPENDIX 1
Surgical observations: Time to hemostasis

| Sheep number | Nostril | Product used | Time to hemostasis (sec) |
|---|---|---|---|
| 1 | LEFT | P | 58 |
| 1 | RIGHT | F | 95 |
| 2 | LEFT | P | 131 |
| 2 | RIGHT | F | 135 |
| 3 | LEFT | P | 87 |
| 3 | RIGHT | F | 115 |
| 4 | LEFT | P | 120 |
| 4 | RIGHT | F | 120 |
| 5 | LEFT | P | 102 |
| 5 | RIGHT | C | 190 |
| 6 | LEFT | F | 140 |
| 6 | RIGHT | C | 285 |
| 7 | RIGHT | F | 145 |
| 7 | LEFT | C | 310 |
| 8 | RIGHT | P | 125 |
| 8 | LEFT | F | 250 |
| 9 | LEFT | P | 130 |
| 9 | RIGHT | F | 210 |
| 10 | LEFT | P | 140 |
| 10 | RIGHT | C | 280 |
| 11 | RIGHT | F | 84 |
| 11 | LEFT | P | 150 |
| 12 | RIGHT | P | 70 |
| 12 | LEFT | F | 115 |
| 13 | RIGHT | P | 130 |
| 13 | LEFT | P | 220 |
| 14 | LEFT | P | 85 |
| 14 | RIGHT | P | 180 |
| 15 | LEFT | C | 180 |
| 15 | RIGHT | C | 200 |
| 16 | LEFT | P | 190 |
| 16 | RIGHT | P | 210 |
| 17 | RIGHT | F | 102 |
| 17 | LEFT | C | 168 |
| 18 | RIGHT | F | 114 |
| 18 | LEFT | C | 240 |
| 19 | LEFT | P | 95 |
| 19 | RIGHT | C | 332 |
| 20 | LEFT | F | 115 |
| 20 | RIGHT | C | 256 |
| 21 | RIGHT | C | 163 |
| 21 | LEFT | F | 164 |
| 22 | LEFT | F | 150 |
| 22 | RIGHT | C | 230 |
| 23 | RIGHT | F | 128 |
| 23 | LEFT | P | 130 |
| 24 | LEFT | C | 107 |
| 24 | RIGHT | P | 290 |
| 25 | LEFT | P | 150 |
| 25 | RIGHT | C | 160 |
| 26 | RIGHT | C | 71 |
| 26 | LEFT | F | 129 |
| 27 | RIGHT | C | 269 |
| 27 | LEFT | F | 326 |
| 28 | RIGHT | F | 100 |
| 28 | LEFT | C | 275 |
| 29 | RIGHT | F | 170 |
| 29 | LEFT | C | 197 |
| 30 | RIGHT | C | 257 |
| 30 | LEFT | C | 310 |

APPENDIX 2
Clinical observations

| | | | Adhesion score | Adhesion score |
|---|---|---|---|---|
| Sheep number | Nostril | Product used | day 14 | day 42 |
| 1 | LEFT | P | 0 | 0 |
| 1 | RIGHT | F | 0 | 0 |
| 2 | LEFT | P | 0 | 0 |
| 2 | RIGHT | F | 4 | 4 |
| 3 | LEFT | P | 0 | 0 |
| 3 | RIGHT | F | 0 | 0 |
| 4 | LEFT | P | 0 | 0 |
| 4 | RIGHT | F | 0 | 0 |
| 5 | LEFT | P | 0 | 0 |
| 5 | RIGHT | C | 4 | 0 |
| 6 | LEFT | F | 4 | 0 |
| 6 | RIGHT | C | 1 | 0 |
| 7 | RIGHT | F | 4 | 4 |
| 7 | LEFT | C | 4 | 4 |
| 8 | RIGHT | P | 0 | 0 |
| 8 | LEFT | F | 0 | 4 |
| 9 | LEFT | P | 0 | 0 |
| 9 | RIGHT | F | 4 | 4 |
| 10 | LEFT | P | 0 | 0 |
| 10 | RIGHT | C | 4 | 4 |
| 11 | RIGHT | F | 4 | 4 |
| 11 | LEFT | P | 0 | 0 |
| 12 | RIGHT | P | 1 | 0 |
| 12 | LEFT | F | 4 | 4 |
| 13 | RIGHT | P | 0 | 0 |
| 13 | LEFT | P | 1 | 0 |
| 14 | LEFT | P | 0 | 0 |
| 14 | RIGHT | P | 0 | 0 |
| 15 | LEFT | C | 0 | 4 |
| 15 | RIGHT | C | 0 | 0 |
| 16 | LEFT | P | 2 | 0 |
| 16 | RIGHT | P | 0 | 0 |
| 17 | RIGHT | F | 4 | 4 |
| 17 | LEFT | C | 4 | 4 |
| 18 | RIGHT | F | 4 | 1 |
| 18 | LEFT | C | 4 | 1 |
| 19 | LEFT | P | 0 | 0 |
| 19 | RIGHT | C | 0 | 0 |
| 20 | LEFT | F | 4 | 4 |
| 20 | RIGHT | C | 4 | 4 |
| 21 | RIGHT | C | 4 | 0 |
| 21 | LEFT | F | 4 | 0 |
| 22 | LEFT | F | 2 | 0 |
| 22 | RIGHT | C | 4 | 0 |
| 23 | RIGHT | F | 3 | 0 |
| 23 | LEFT | P | 0 | 0 |
| 24 | LEFT | C | 0 | 0 |
| 24 | RIGHT | P | 0 | 0 |
| 25 | LEFT | P | 1 | 0 |
| 25 | RIGHT | C | 4 | 0 |
| 26 | RIGHT | C | 4 | 0 |
| 26 | LEFT | F | 4 | 0 |
| 27 | RIGHT | C | 4 | 0 |
| 27 | LEFT | F | 4 | 0 |
| 28 | RIGHT | F | 4 | 0 |
| 28 | LEFT | C | 4 | 0 |
| 29 | RIGHT | F | 4 | 1 |
| 29 | LEFT | C | 4 | 1 |
| 30 | RIGHT | C | 0 | 0 |
| 30 | LEFT | C | 4 | 0 |

APPENDIX 3
Graded Histological Samples

| Sheep | | Product | | | | | | |
|---|---|---|---|---|---|---|---|---|
| number | Nostril | used | EE | II | SF | A | SMC | GC |
| 1 | LEFT | P | 2 | 1 | 1 | 1 | −1 | 1 |
| 1 | RIGHT | F | 3 | 1 | 3 | 2 | 1 | 0 |
| 2 | LEFT | P | 2 | 1 | 1 | 1 | 0 | 0 |
| 2 | RIGHT | F | 2 | 1 | 2 | 1 | −1 | −1 |
| 3 | LEFT | P | 1 | 2 | 1 | 1 | −1 | −1 |
| 3 | RIGHT | F | 3 | 1 | 3 | 3 | 0 | 0 |
| 4 | LEFT | P | 2 | 2 | 2 | 3 | 0 | 1 |
| 4 | RIGHT | F | 2 | 2 | 3 | 1 | 0 | 1 |
| 5 | LEFT | P | 1 | 1 | 1 | 1 | 0 | 0 |
| 5 | RIGHT | C | 3 | 1 | 2 | 1 | 0 | 0 |
| 6 | LEFT | F | 2 | 2 | 1 | 0 | −1 | 0 |
| 6 | RIGHT | C | 2 | 2 | 3 | 2 | −1 | 0 |
| 7 | RIGHT | F | 3 | 2 | 3 | 1 | −1 | 0 |
| 7 | LEFT | C | 3 | 1 | 2 | 0 | −1 | 0 |
| 8 | RIGHT | P | 2 | 1 | 1 | 0 | 0 | 1 |
| 8 | LEFT | F | 1 | 2 | 3 | 2 | −1 | 0 |
| 9 | LEFT | P | 2 | 0 | 1 | 0 | 0 | −1 |
| 9 | RIGHT | F | 3 | 1 | 3 | 3 | −1 | 0 |
| 10 | LEFT | P | 2 | 2 | 1 | 2 | 0 | 1 |
| 10 | RIGHT | C | 2 | 2 | 2 | 1 | −1 | 1 |
| 11 | RIGHT | F | 1 | 2 | 3 | 2 | −1 | −1 |
| 11 | LEFT | P | 1 | 1 | 1 | 1 | 0 | 0 |
| 12 | RIGHT | P | 1 | 2 | 1 | 0 | 0 | −1 |
| 12 | LEFT | F | 2 | 1 | 3 | 3 | −1 | −1 |
| 13 | RIGHT | P | 1 | 1 | 1 | 0 | 0 | −1 |
| 13 | LEFT | P | 1 | 0 | 0 | 0 | −1 | −1 |
| 14 | LEFT | P | 0 | 1 | 0 | 0 | 0 | 1 |
| 14 | RIGHT | P | 0 | 1 | 1 | 0 | 0 | −1 |
| 15 | LEFT | C | 0 | 1 | 2 | 2 | −1 | 0 |
| 15 | RIGHT | C | 3 | 1 | 3 | 3 | −1 | 0 |
| 16 | LEFT | P | 1 | 1 | 2 | 2 | −1 | 1 |
| 16 | RIGHT | P | 1 | 1 | 2 | 2 | −1 | 0 |
| 17 | RIGHT | F | 2 | 0 | 1 | 0 | 0 | 0 |
| 17 | LEFT | C | 2 | 1 | 3 | 1 | −1 | 0 |
| 18 | RIGHT | F | 0 | 2 | 2 | 2 | 0 | 0 |
| 18 | LEFT | C | 1 | 2 | 3 | 2 | −1 | −1 |
| 19 | LEFT | P | 3 | 1 | 3 | 2 | −1 | −1 |
| 19 | RIGHT | C | 1 | 2 | 3 | 3 | −1 | 0 |
| 20 | LEFT | F | 3 | 1 | 3 | 1 | −1 | −1 |
| 20 | RIGHT | C | 3 | 2 | 3 | 3 | −1 | −1 |
| 21 | RIGHT | C | 2 | 1 | 3 | 2 | −1 | −1 |
| 21 | LEFT | F | 3 | 1 | 3 | 3 | −1 | −1 |
| 22 | LEFT | F | 2 | 1 | 1 | 0 | 0 | 0 |
| 22 | RIGHT | C | 0 | 1 | 2 | 1 | 0 | −1 |
| 23 | RIGHT | F | 1 | 1 | 3 | 3 | −1 | −1 |
| 23 | LEFT | P | 0 | 1 | 0 | 0 | 0 | −1 |
| 24 | LEFT | C | 0 | 1 | 1 | 0 | 0 | 0 |
| 24 | RIGHT | P | 0 | 0 | 0 | 0 | 0 | 0 |
| 25 | LEFT | P | 0 | 1 | 1 | 1 | 0 | 0 |
| 25 | RIGHT | C | 0 | 1 | 1 | 1 | 0 | −1 |
| 26 | RIGHT | C | 0 | 1 | 1 | 0 | 0 | 0 |
| 26 | LEFT | F | 1 | 1 | 3 | 2 | −1 | 0 |
| 27 | RIGHT | C | 3 | 2 | 3 | 2 | −1 | −1 |
| 27 | LEFT | F | 0 | 1 | 3 | 3 | 0 | 0 |
| 28 | RIGHT | F | 0 | 1 | 2 | 2 | −1 | 0 |
| 28 | LEFT | C | 1 | 1 | 3 | 3 | 0 | 0 |
| 29 | RIGHT | F | 0 | 2 | 3 | 3 | −1 | −1 |
| 29 | LEFT | C | 0 | 3 | 1 | 1 | 0 | 0 |
| 30 | RIGHT | C | 2 | 0 | 3 | 2 | −1 | 0 |
| 30 | LEFT | C | 0 | 1 | 2 | 2 | 0 | 0 |

We claim:

1. A method of reducing negative sequelae from endoscopic turbinoplasty or FESS in a subject, the method comprising applying an effective amount of one or more self-assembling peptides (SAP) in solution, wherein the peptide is chosen from Table 1, said application taking place during or immediately after an endoscopic turbinoplasty or FESS wherein the SAP solution is applied via a catheter to the surgically affected area in the turbinate, thereby forming a transparent, resorbable viscous gel in contact with the wound, thereupon reducing negative sequelae from endoscopic turbinoplasty or FESS of said subject.

2. The method of claim 1, wherein the peptide is chosen from: a) SEQ NO:1 at a purity of at least 65% in water or physiological buffer and/or at a concentration of between 1.5 and 3.0% weight/volume and b) SEQ ID NO:3 at a major peptide purity of at least 70% in water or physiological buffer and at a concentration of total peptide between 1.5% and 3.5% weight/volume.

3. The method of claim 1, where post-surgical synechiae formation in turbine(s) and/or sinus orifice(s) is less than 25%, as compared to untreated controls.

3. The method of claim 1, where healing over a period of weeks after the endoscopic turbinoplasty or FESS, improves in one of more the following observable metrics:
A. Reduction in epithelial erosion
B. Reduction in inflammatory infiltrate
C. Reduction in submucosal fibrosis
D. Improved angiogenesis
E. Presence/absence of seromucous glands
F. Presence/absence of goblet cells

4. The method of claim 1, wherein the bleeding stops within several minutes from the administration of the self-assembling peptide solution to the surgical site without the use of other hemostatic agents, techniques, or instruments.

5. The method of claim 1, wherein the SAP solution is applied to one or more sinus orifices, thereby preventing formation of “clogging” or blocking synechiae that may close the sinus air circulation and create an environment for chronic sinus infection.

6. The method of claim 4, wherein post-surgical or delayed bleeding from the surgical site does not occur.

7. The method of claim 1, wherein, even when post-operative synechiae occur, the subject's condition is resolved atraumatically.

8. The method of claim 1, wherein the subject does not report pain or discomfort resulting from the surgical procedure.


SEQUENCE LISTING (23 sequences)

SEQ ID NO: 1 - protein, 16 aa, synthetic construct
  REGION 1..16: note = Description of Artificial Sequence: Synthetic Peptide
RADARADARADARADA

SEQ ID NO: 2 - protein, 12 aa, synthetic construct
  REGION 1..12: note = Description of Artificial Sequence: Synthetic Peptide
KLDKLDKLDKLD

SEQ ID NO: 3 - protein, 13 aa, synthetic construct
  REGION 1..13: note = Description of Artificial Sequence: Synthetic Peptide
IEIKIEIKIEIKI

SEQ ID NO: 4 - protein, 12 aa, synthetic construct
  REGION 1..12: note = Description of Artificial Sequence: Synthetic Peptide
QLELQLELQLEL

SEQ ID NO: 5 - protein, 16 aa, synthetic construct
  REGION 1..16: note = Description of Artificial Sequence: Synthetic Peptide
ADARADARADARADAR

SEQ ID NO: 6 - protein, 16 aa, synthetic construct
  REGION 1..16: note = Description of Artificial Sequence: Synthetic Peptide
RAEARAEARAEARAEA

SEQ ID NO: 7 - protein, 16 aa, synthetic construct
  REGION 1..16: note = Description of Artificial Sequence: Synthetic Peptide
RVDVRVDVRVDVRVDV

SEQ ID NO: 8 - protein, 16 aa, synthetic construct
  REGION 1..16: note = Description of Artificial Sequence: Synthetic Peptide
RLDLRLDLRLDLRLDL

SEQ ID NO: 9 - protein, 16 aa, synthetic construct
  REGION 1..16: note = Description of Artificial Sequence: Synthetic Peptide
RIDIRIDIRIDIRIDI

SEQ ID NO: 10 - protein, 16 aa, synthetic construct
  REGION 1..16: note = Description of Artificial Sequence: Synthetic Peptide
RFDFRFDFRFDFRFDF

SEQ ID NO: 11 - protein, 16 aa, synthetic construct
  REGION 1..16: note = Description of Artificial Sequence: Synthetic Peptide
AEARAEARAEARAEAR

SEQ ID NO: 12 - protein, 16 aa, synthetic construct
  REGION 1..16: note = Description of Artificial Sequence: Synthetic Peptide
KADAKADAKADAKADA

SEQ ID NO: 13 - protein, 16 aa, synthetic construct
  REGION 1..16: note = Description of Artificial Sequence: Synthetic Peptide
KIDIKIDIKIDIKIDI

SEQ ID NO: 14 - protein, 16 aa, synthetic construct
  REGION 1..16: note = Description of Artificial Sequence: Synthetic Peptide
KIEIKIEIKIEIKIEI

SEQ ID NO: 15 - protein, 13 aa, synthetic construct
  REGION 1..13: note = Description of Artificial Sequence: Synthetic Peptide
IDIKIDIKIDIKI

SEQ ID NO: 16 - protein, 13 aa, synthetic construct
  REGION 1..13: note = Description of Artificial Sequence: Synthetic Peptide
IEIRIEIRIEIRI

SEQ ID NO: 17 - protein, 13 aa, synthetic construct
  REGION 1..13: note = Description of Artificial Sequence: Synthetic Peptide
LELKLELKLELKL

SEQ ID NO: 18 - protein, 13 aa, synthetic construct
  REGION 1..13: note = Description of Artificial Sequence: Synthetic Peptide
FEFKFEFKFEFKF

SEQ ID NO: 19 - protein, 12 aa, synthetic construct
  REGION 1..12: note = Description of Artificial Sequence: Synthetic Peptide
KLDLKLDLKLDL

SEQ ID NO: 20 - protein, 12 aa, synthetic construct
  REGION 1..12: note = Description of Artificial Sequence: Synthetic Peptide
KLELKLELKLEL

SEQ ID NO: 21 - protein, 13 aa, synthetic construct
  REGION 1..13: note = Description of Artificial Sequence: Synthetic Peptide
FEFRFEFRFEFRF

SEQ ID NO: 22 - protein, 13 aa, synthetic construct
  REGION 1..13: note = Description of Artificial Sequence: Synthetic Peptide
YEYKYEYKYEYKY

SEQ ID NO: 23 - protein, 13 aa, synthetic construct
  REGION 1..13: note = Description of Artificial Sequence: Synthetic Peptide
WEWKWEWKWEWKW